(19) United States
(12) Patent Application Publication
(10) Pub. No.: US 2026/0204419 A1
(43) Pub. Date: Jul. 16, 2026

(54) CLINICAL ANALYSIS AND METHODS FOR PREDICTING THE RISK OF DEVELOPING CLINICALLY SIGNIFICANT PROSTATE CANCER

(71) Applicant: Nanostics Inc., Edmonton (CA)

(72) Inventors: John Lewis, Edmondton (CA); Robert Paproski, Edmonton (CA)

(21) Appl. No.: 19/132,722

(22) PCT Filed: Nov. 3, 2023
(86) PCT No.: PCT/CA2023/051470

§ 371 Date: May 23, 2025

(30) Foreign Application Priority Data
Nov. 4, 2022 (CA) 3181305

(51) Int. Cl. G16H 50/30; G06N 20/00; G16H 10/60; G16H 20/00; G16H 50/20; G16H 50/70

(52) U.S. Cl. CPC G16H 50/30; G06N 20/00; G16H 10/60; G16H 20/00; G16H 50/20; G16H 50/70

(57) ABSTRACT

This invention relates to a method, a non-transitory computer readable medium and a system for predicting the risk of developing clinically significant prostate cancer. A plurality of patients are selected based on a set of pre-determined criteria and clinical data points are obtained from the patients. The collected data points are further processed, engineered or statistically converted and the model hyper parameters are optimized by grid searching. The processed data points and the optimized hyper parameters are fed into a prediction system that is trained to generate a risk score using the information provided. The risk or probability of developing clinically significant prostate cancer is predicted based on the risk score.

### FIELD OF THE INVENTION

This invention relates to a method, a non-transitory computer readable medium and a kit for predicting clinically significant data for prostate cancer analysis. More specifically, the invention relates to a neural network model, an optimized random forest model, and a prediction system that is capable of calculating the risk of a patient developing clinically significant prostate cancer.

### BACKGROUND

Prostate cancer is the most diagnosed cancer in men and the second leading cause of male cancer deaths in the United States. Currently, the prostate-specific antigen (PSA) blood test is used to screen men for prostate cancer before a biopsy. However, the US Preventive Services Task Force (USPSTF) recommends against PSA screening because of its low specificity for Group Grade (GG)≥2 prostate cancer leading to the over-diagnosis of indolent prostate cancer. Over-diagnosis is associated with over-treatment, exposing patients to potential adverse effects and unnecessary healthcare costs. Therefore, there is a critical need for more specific prostate cancer tests to counter the overdiagnosis of low-grade prostate cancer identified from PSA testing.

Risk models for prostate cancer include multivariable clinical predictors such as Prostate Cancer Prevention Trial Risk Calculator (PCPTRC), European Randomized Screening for Prostate Cancer (ERSPC), Prostate Biopsy Collaborative Group (PBCG), and diagnostic biomarker tests such as 4Kscore and PHI. Many studies suggest that MRI with targeted biopsy is better than systematic biopsies for making a correct diagnosis of clinically significant prostate cancer but is also highly dependent on the level of expertise of the operator and pathologist. There are substantial intra-reader, and inter-reader variability with mpMRI outputs, making them not as readily reproducible as biofluid test results, and MRI still misses some men with clinically significant prostate cancer. Some reports estimate that the annual cost of routine mpMRI-use prior to biopsy in the US would be $3 billion, roughly 15% of the entire cost of managing prostate cancer.

Prostate cancer is the second most diagnosed cancer in men, with an estimated 1.4 million diagnoses worldwide in 2020. In Europe, prostate cancer is the most common male cancer, with an estimated 450000 new cases and 107000 deaths reported in 2018. The geographical incidence of prostate cancer diagnosis varies widely, being highest in Australia, New Zealand, North America, and Western and Northern Europe, largely due to the use of Prostate Specific Antigen (PSA) testing and the aging population. In comparison, Eastern and South-Central Asia, and Eastern and Southern Europe show a low incidence of prostate cancer diagnosis, although rates are steadily increasing in these locations. Conversely, over the last 20 years, the age-standardized mortality rate has been decreasing. This steady decline began shortly after the peak incidence of prostate cancer diagnosis occurred, around 1993, when the PSA test became available as a common laboratory test. PSA is the current prostate cancer screening biomarker, and at the standard PSA level cut-off (3 ng/ml) has a high negative predictive value for prostate cancer, which is the probability that a negative test indicates the absence of disease. However, PSA has low specificity for prostate cancer because men with non-cancerous prostate diseases such as prostatitis or benign hyperplasia also show elevated PSA levels. The use of PSA as a screening tool led to the costly over-diagnosis and overtreatment of prostate cancer. The European prostate cancer guidelines developed by a group of urologists, radiation oncologists, medical oncologists, radiologists, a pathologist, a geriatrician and a patient representative, recommend against systematic screening with the PSA test but states an increased interest in early individualized detection. The EAU guidelines stress the need to break the link between diagnosis and active treatment to decrease over-treatment, while still offering early diagnosis for men who request.

Patient risk stratification methods to determine the need for a prostate biopsy to establish the diagnosis of prostate cancer vary regionally. However, elevated PSA levels and a digital rectal examination (DRE) finding of abnormal is often the trigger for biopsy referral. The prostate biopsy procedure is invasive and painful and may cause anxiety, stress, depression, fever, rectal bleeding, urinary obstruction, infections, sepsis, and myocardial infarctions. These harms can significantly affect the patients' quality of life and may also reduce their quality-adjusted life years (QALYs). The overdiagnosis of prostate cancer in men who would not have developed clinically significant prostate cancer in their lifetime is associated with overtreatment with aggressive therapies that may cause adverse effects, such as impotence and incontinence, and unnecessary healthcare costs.

While the five-year relative survival for localized prostate cancer is 95% for Canada and 99% for the USA, the 5-year relative survival drops to 31% for patients presenting with distant prostate cancer. Other important clinical prognostic factors include age, comorbidities, pathologic indices of aggressiveness, and Gleason grade group and stage (locally advanced disease with invasion of seminal vesicles). Morbidity and mortality of prostate cancer are consequences of the biomolecular processes that lead to metastatic dissemination, which is determined by a tumour cell's ability to enter the stroma, invade the surrounding vasculature, travel to a distant site, and grow in a new microenvironment.

The myriad of biological processes that drive prostate cancer progression provides the rationale for research to identify important biomarkers of metastatic potential during the latent or early phases of cancer development. Growing evidence suggests that low-risk patients is manageable with “active surveillance” defined as periodic assessment and continued observation of patients with low-risk, non-progressive localized prostate cancer. Opportunities are now available for studying the natural history of the disease, and, in turn, to search for biomarkers that may distinguish low-risk from high-risk patients. Such information would be invaluable to helping clinicians and patients decide on an appropriate risk-based management strategy.

The European Association of Urologists (EAU) guidelines for the early detection of prostate cancer list the currently available urine, serum and tissue-based biomarkers proposed for improving detection and risk stratification of prostate cancer patients, and potentially avoiding unnecessary biopsies. However, the guidelines also suggest that further studies are necessary to validate their efficacy as the data is too limited to implement these markers into routine screening programs. Therefore, at present EAU prostate cancer early detection guidelines weakly recommend the use of other biomarker tests, in addition to PSA, to improve the sensitivity and specificity for screening of high-grade prostate cancer. The EAU guidelines also outlines that risk calculators, including clinical information such as age, DRE findings, PSA levels, and other data, may be useful to determine an individual's risk of high-grade prostate cancer, thereby reducing the number of unnecessary biopsies.

The US National Comprehensive Cancer Network (NCCN) guidelines for the early detection of prostate cancer outline that although the use of biomarkers in conjunction with serum PSA levels to improve the specificity of detection is not yet mandated, there may be patients that would benefit from further defining prostate cancer risk with the use of biomarker tests. NCCN recommends that a patient with a PSA level of >3 ng/ml undergoes a pre-biopsy workup that includes a repeat PSA and a DRE, and that biomarkers to improve the screening specificity should be considered before a biopsy.

The EAU prostate cancer guidelines panel recommends against population-based PSA screening because of evidence consistent with greater harms than benefits. However, the uncertainties around lifetime probabilities of a common condition cannot be easily understood to dissuade tendencies towards screening.

As health and life expectancy concerns permeate societies confronting an aging “baby-boomer” population, there is a pressing need to identify “a better test and better treatment options” for the management of prostate cancer. Over diagnosis of prostate cancer leads to many asymptomatic men with indolent prostate cancer or non-cancer prostate disease undergoing unnecessary prostate biopsies. These numbers illustrate the need for a non-invasive test that can accurately predict clinically significant prostate cancer and stratify it to dictate treatment options. Therefore, there is a critical need for a prostate cancer screening test that uses prostate cancer-specific biomarkers to counter the over diagnosis of localized, non-clinically significant prostate cancer identified from PSA testing.

While the reduction of prostate cancer mortality is attributed to the advent of therapies such as surgery, radiotherapy, and hormone treatments that are employed to manage prostate cancer, there remains a fundamental problem of over-diagnosis due to PSA screening. Over-diagnosis results in patients being informed that they have prostate cancer when they do not have clinically significant prostate cancer, and this is also often associated with overtreatment. Therefore, stratifying prostate cancer patients into low or high-risk groups during the initial diagnosis is a major clinical need to be addressed.

### SUMMARY OF THE INVENTION

In one embodiment of the invention, a Software as a Medical Device (SaMD), non-transitory computer readable medium (CRM) and/or method for predicting a possibility of developing clinically significant prostate cancer is provided by obtaining one or more clinical data points of a plurality of patients, statistically converting one or more clinical data points before feeding them into a prediction system, training the prediction system using a software module, where the software module trains the prediction system to create a probability model using the one or more statistically converted clinical data points, and predicting the possibility of developing clinically significant prostate cancer using the probability model created by the prediction system. The probability model can be a logistic regression model or a neural network model.

In some embodiments, the prediction system is a computer, an operating system, a software, or a machine learning algorithm where the software module that trains the prediction system to create the probability model is a Tensorflow module or a Python's scikit-learn module. The software module that trains the prediction system to create the neural network model is the Tensorflow module. The software module that trains the prediction system to create the logistic regression model is the Python's scikit-learn module which comprises a Python's scikit-learn library software module, Python's pandas software module, Python's numpy software module, Python's onnx software module, Python's onnxruntime software module, Python's onnxmltools software module, and Python's onnxconverter_common software module.

The prediction system described herein can be trained using a variety of optimization algorithms and deep learning methods available in the art. For instance, the algorithms can be optimized by designing a training problem with a plurality of trainable parameters and could be trained using various optimization techniques, some non-limiting examples of which are Gradient descent method, Newton method, Stochastic, descent method, Mini-batch gradient method, Nesterov accelerated gradient method, Adagrad, AdaDelta, Adaptive moment estimation, Conjugate gradient method, Quasi-Newton method, and Levenberg-Marquardt algorithm.

A person skilled in the art would understand that alternative techniques such as MATLAB, IBM Watson Studio, Google Cloud AI Platform, Theano, PyTorch, OpenCV, Keras, Apache Spark, Amazon SageMaker, Google Cloud AutoML, RapidMiner, Azure Machine Learning Studio, and Anaconda could also be employed.

In the embodiments where Python's scikit-learn module is required, the module trains the prediction system system by optimizing a set of model hyperparameters to obtain a favorable AUC ROC value using the one or more clinical data points, wherein the model hyperparameters are optimized by grid searching through the model hyperparameters individually and/or in combination. The model hyperparameters are usually selected from Penalty value, tol, C value or solver, where the Penalty value is 11 or 12; where the total value is 1e−4, where the C value is 0.1, 0.25, 0.5, 1.0, 2.5, 5, 10, 25, 50, 100, 250, 500, or 1 000; and where the solver value is liblinear. The Python's onnxmltools software module converts the probability model of the prediction system to ONNX format. In an alternate embodiment, a kit for predicting a possibility of developing clinically significant prostate cancer is provided that comprises a dataset of one or more clinical data points, means for statistically converting the one or more data points, a prediction system configured to create a probability model using the one or more clinical data points or the one or more statistically converted data points; and a software module configured to train the prediction system to create the probability model using the one or more clinical data points or the one or more statistically converted data points.

In another embodiment of the invention, a method for predicting the risk of developing clinically significant prostate cancer is provided. The method involves processing one or more clinical data points that are obtained from a plurality of patients. The patients are selected based on a set of pre-determined criteria. The set of pre-determined criteria could be any patient feature or characteristic that may be related or have any significance on the prostate cancer diagnosis or prognosis. In some embodiments, the pre-determined criteria could be patient age, prior prostate cancer diagnosis, referral for biopsy, total prostate specific antigen (total PSA) result, prostate volume or MRI prostate volume, digital rectal exam result, PI-RADS score or a combination of any one of the above.

The method also involves optimization of one or more model hyper parameters by a grid searching technique. The model hyper parameters could be any statistical formula or standardization technique that would help improve or optimize the analysis of the one or more clinical data points or optimize the risk prediction capability of the proposed technique.

The processed, standardized or engineered clinical data points are fed into a prediction system along with the optimized model hyper parameters. The prediction system is the trained using a machine learning or software module or a training package that trains the prediction system to generate a risk score using the processed clinical data points and the model hyper parameters. The trained prediction system predicts the risk of developing clinically significant prostate cancer based on the generated or calculated risk score generated. If the risk score is high, the prediction system may recommend that the patient seek immediate clinical evaluation, biopsy and/or medical intervention; and if the risk score is low the system may recommend future clinical evaluation or medical intervention if needed.

The invention also provides a non-transitory computer readable medium having stored thereon an algorithm that, when executed by a processor, cause the processor to perform the method for predicting the risk of developing clinically significant prostate cancer, wherein the algorithm performs the method as recited above. The invention also provides kits, software module and a system for predicting the risk of clinically significant prostate cancer.

### BRIEF DESCRIPTION OF DRAWINGS

FIG. 1 shows a flow-chart explaining the SaMD/CRM based probability model in a real-world setting.

FIG. 2 shows a risk discussion chart between physician and patients weighing in on life expectancy and co-morbidities depending on the SaMD/CRM risk assessment.

FIG. 3 shows the Proof of Concept Test AUCs as conducted using an initial 1,437 patient cohort.

FIG. 4 shows a comparison chart of ROC AUC values of the default and optimized models.

FIG. 5 shows the probability of grade group 2 prostate cancer based on free PSA ratio.

FIG. 6 shows the probability of grade group 2 prostate cancer based on age of the patient.

FIG. 7 shows a comparison of 11 machine learning algorithms for predicting grade group ≥2 prostate cancer using the receiver operating characteristic area under the curve (ROC AUC) values. ROC AUC values were compared using DeLong's method. In the figure, the initialism's are as follows: GBM: gradient-boosting machine, DA: discriminant analysis, RBF: radial basis function, SVM: support vector machines, KNN: k-nearest neighbors.

FIG. 8 shows the correlation between age and grade group ≥2 prostate cancer probability in the train cohort. An exponential growth equation has a higher R squared value than linear regression when fitting age and probability of grade group ≥2 prostate cancer.

FIG. 9 shows optimized neural network model has improved receiver operating characteristic area under the curve (ROC AUC) values over default logistic regression models for predicting grade group ≥2 prostate cancer in training and validation cohorts. Receiver characteristic curve area under the curve (ROC AUC) values were compared using DeLong's method.

FIG. 10 shows feature selection by feature elimination for models predicting grade group ≥2 prostate cancer. Free PSA ratio was the most important feature for all models since its removal caused the largest decrease in model receiver operating characteristic area under the curve (ROC AUC). ROC AUC values were compared to the inclusion of all features using DeLong's method. GG≥2 PCa, grade group ≥2 prostate cancer, DRE: digital rectal exam findings (normal or abnormal), PNB: prior negative biopsies status (yes or no), PSA: prostate specific antigen.

FIG. 11 shows receiver operating characteristic curves for the proposed technique (hereinafter referred to as “ClarityDX Prostate”), PBCG, % Free PSA, and PSA when predicting grade group ≥2 prostate cancer in the training cohort (A) and validation cohort (B). Area under the curve (AUC) values were compared using DeLong's method. ClarityDX Prostate had significantly greater than AUC values than all other tested risk calculators and PSA for predicting prostate cancer and grade group ≥2 prostate cancer in the training and validation cohorts. Sen: sensitivity, Spe: specificity.

FIG. 12 shows receiver operating characteristic curves for ClarityDX Prostate, PHI, PBCG, % Free PSA, and PSA when predicting grade group ≥2 prostate cancer for Thomayer University Hospital patients.

FIGS. 13A and 13B shows calibration curves for ClarityDX Prostate model in the train (A) and validation (B) cohorts when predicting grade group ≥2 prostate cancer. Data points fitted with linear regression with R squared values and Brier scores shown. Calibration curves trend close to the perfect calibration line for training and validation cohorts. Linear regression equations fit near the perfectly calibrated line with R squared values of 0.997 and 0.987 for the train and validation cohorts, respectively.

FIG. 14 shows decision curve analysis of ClarityDX Prostate, PBCG, % free PSA, and PSA for predicting grade group ≥2 prostate cancer in the train (A, C) and validation (B, D) cohorts. Net benefit (A, B) and net avoided interventions per 100 patients (C, D) were analyzed assuming interventions required for grade group ≥2 prostate cancer. For almost all probability thresholds, ClarityDX Prostate outperforms the other tests. The recommended ≥25% ClarityDX Prostate threshold is well within the area of highest net benefit for identifying high-risk patients for GG ≥2 prostate cancer.

### DETAILED DESCRIPTION

The following description is of preferred embodiments by way of example only and without limitation to the combination of features necessary for carrying the invention into effect.

All terms are intended to be understood as they would be understood by a person skilled in the art. Unless defined otherwise, all technical and scientific terms used herein have the same meaning as commonly understood by one of ordinary skill in the art to which the disclosure pertains. The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application. Accordingly, the terminology used herein is for the purpose of describing particular embodiments only, and is not intended to be limiting.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof and their grammatical equivalents as used herein, can be used interchangeably. These terms can convey that any and all combinations are specifically contemplated. The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” “alternate embodiment”, or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value. The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11.

The initialism “GG” as used in the descriptive embodiments mean “Grade Group”.

The initialism “PCa” or “PCA” as used in the descriptive embodiments mean “Prostate cancer”.

The initialism “ROC AUC” as used in the descriptive embodiments mean “Receiver operating characteristic area under the curve”.

The initialism “CI” as used in the descriptive embodiments mean “Confidence interval (95%)”.

The initialism “PPV” as used in the descriptive embodiments mean “Positive predictive value”.

The initialism “NPV” as used in the descriptive embodiments mean “Negative predictive value”. The initialism “SD” as used in the descriptive embodiments mean “Standard deviation”.

The initialism “DRE” as used in the descriptive embodiments mean “Digital rectal exam”.

The initialism “IQR” as used in the descriptive embodiments mean “Interquartile range”.

The initialism “PSA” as used in the descriptive embodiments mean “Prostate-specific antigen”.

The initialism “ERSPC-3” as used in the descriptive embodiments mean “European Randomized study of Screening for Prostate Cancer risk calculator 3”.

The initialism “PCPTRC 2.0” as used in the descriptive embodiments mean “Prostate Cancer Prevention Trial Risk Calculator 2.0”.

The initialism “PBCG” as used in the descriptive embodiments mean “Prostate Biopsy Collaborative Group risk calculator”.

The initialism “CV” as used in the descriptive embodiments mean “Coefficient of Variability”.

The initialism “LDT” as used in the descriptive embodiments mean “Laboratory Developed Test”.

The initialism “NAS” as used in the descriptive embodiments mean “Network Attached Storage”.

The initialism “fPSA” as used in the descriptive embodiments mean “Free Prostate Specific Antigen”.

The initialism “POC” as used in the descriptive embodiments mean “Proof of Concept”.

The initialism “SST” as used in the descriptive embodiments mean “Serum Separation Tubes”.

The initialism “SaMD” as used in the descriptive embodiments refer to “Software as a Medical Device” as proposed in the present invention.

The initialism “CRM” as used in the descriptive embodiments refer to “Non-transitory Computer Readable Medium” as proposed in the present invention.

The initialism “CV” as used in the descriptive embodiments mean “Coefficient of Variability”.

The expression “clinically significant prostate cancer” means prostate cancer that is grade group 2 or greater.

The expression “prediction system” is a software as a medical device that is capable of calculating the risk of having clinically significant prostate cancer using well-defined and trained algorithms.

The windows-based computer systems referred to in the present description relate to analysis of analytical and clinical data.

In some embodiments, the proposed SaMD/CRM uses the Tensorflow software for clinical performance and analysis. In some alternate embodiments, the proposed SaMD/CRM utilizes a prediction system that employs the following software for clinical performance and analysis:
Python (version 3.8 or higher)
Pandas (Python module for working with tables of data)
Numpy (Python module that manipulates data arrays).
Scikit-learn (Python module for calculating receiver operating characteristic area under the curve (AUC) and confusion matrix for calculating other statistics); and
onnxruntime (Python library for probability model inference).

The software for analytical and clinical performance may also include:
GraphPad Prism software (version 9.3.1 (471) or higher).

### Embodiments Pertaining to First Computer Implemented Method, SaMD and CRM

The present invention relates to a Non-transitory Computer Readable Medium (CRM) or a Software as a Medical Device (SaMD) and provides for methods for analyzing clinically significant data for prostate cancer analysis. More specifically, the invention relates to a software system that is capable of calculating the risk of having clinically significant prostate cancer. In some embodiments, a prediction system is proposed that creates a probability model for determining the possibility of a patient developing clinically significant prostate cancer. The prediction system is designed to create two types of probability models i.e. a neural network model or a logistic regression model; and the one employed depends on the datasets being analyzed.

The proposed SaMD/CRM, also known as ClarityDX Prostate® test is a software as a medical device that calculates the risk of having clinically significant prostate cancer, defined as Gleason grade group 2 or higher, on prostate biopsy. The SaMD/CRM test combines the results of immunoassays and one or more clinical data points to obtain a numerical score, called a Risk Score, that is intended to assist in predicting the presence of clinically significant prostate cancer. In some embodiments, the immunoassays used could be Roche Elecsys total PSA (prostate specific antigen) and/or Roche Elecsys free PSA.

The ClarityDX Prostate® test is designed to meet the clinical need to improve specificity for clinically significant prostate cancer. The test may combine data from any clinically significant prostate cancer diagnostic tests with prostate cancer risk-related clinical information to generate a Risk Score. The ClarityDX Prostate® test is intended as a reflex prostate cancer test to help physicians and patients make an informed decision as to proceed to biopsy or not, after an elevated PSA test result.

The ClarityDX Prostate® test generates machine learning probability models from the patients' clinical information including PSA and fPSA to determine the final probability of patients having clinically significant prostate cancer by calculating the Risk Score. Risk Scores represent probabilities that the patient has low or high risk of developing clinically significant prostate cancer. In addition to the quantitative probability range, the Risk Score is expressed semi-quantitatively as either Low-Risk or High-Risk.

The proposed SaMD/CRM test shares some characteristics in common with other prostate cancer diagnostic tests or risk calculators, such as the use of blood tests, immunogenic detection of prostate cancer biomarkers, patient clinical information as data sets and use of machine learning algorithms to calculate a score for high grade prostate cancer.

In some embodiments, the SaMD/CRM based probability model uses machine learning algorithms to process patient-related clinical information, also known as clinical data points or clinical data features, some non-limiting examples of which are: age, previous biopsy results, number of previous negative prostate biopsies, total PSA levels, free PSA levels, and digital rectal examination (DRE) results; and these clinical data points are used to predict the risk of high-grade prostate cancer and avoid unnecessary biopsies.

In an embodiment of the invention, a non-transitory computer readable medium (CRM) having stored thereon an algorithm that, when executed by a processor, cause the processor to perform the method for predicting a possibility of developing clinically significant prostate cancer is provide. The CRM's algorithm statistically converts one or more clinical data points of a plurality of patients and feeds the one or more statistically converted clinical data points into a prediction system. The prediction system is trained using a software module which trains the prediction system to create a probability model using the one or more statistically converted clinical data points and the CRM's algorithm predicts the possibility of developing clinically significant prostate cancer using the probability model created by the prediction system.

In an alternate embodiment of the invention, a Software as a Medical Device (SaMD) for predicting a possibility of developing clinically significant prostate cancer is provided, where the software is trained to carry out the steps of obtaining one or more clinical data points of a plurality of patients, statistically converting one or more clinical data points before feeding them into a prediction system, training the prediction system using a software module, where the software module trains the prediction system to create a probability model using the one or more statistically converted clinical data points, and predicting the possibility of developing clinically significant prostate cancer using the probability model created by the prediction system.

In a further embodiment of the invention, a method for predicting a possibility of developing clinically significant prostate cancer is provided. The method comprises obtaining one or more clinical data points of a plurality of patients, statistically converting one or more clinical data points before feeding them into a prediction system, training the prediction system using a software module, where the software module trains the prediction system to create a probability model using the one or more statistically converted clinical data points, and predicting the possibility of developing clinically significant prostate cancer using the probability model created by the prediction system.

In some embodiments, the SaMD/CRM or method comprises standardizing the value of one or more clinical data points or of one or more statistically converted clinical data points. In some embodiments, the SaMD/CRM or the method comprises log-transforming one or more clinical data points. In some other embodiments, the method comprises obtaining a ratio of one or more clinical data points.

In some further embodiments, the SaMD/CRM or the method comprises creating the probability model by calculating a Risk Score using the one or more statistically converted clinical data points. The probability model uses the Risk Score to predict the possibility of developing clinically significant prostate cancer, where calculating the Risk Score comprises, recommending immediate clinical evaluation, biopsy and/or medical intervention if the Risk Score is high or recommending future clinical evaluation and/or medical intervention if the Risk Score is low.

In some embodiments, the SaMD/CRM or method further comprises the step of testing the probability model created by the prediction system for accuracy using one or more pre-defined metric, where the one or more pre-defined metric is diagnostic specificity, diagnostic sensitivity, diagnostic positive predictive value, diagnostic negative predictive value, or a combination thereof.

In some further embodiments, the SaMD/CRM or method involves grid searching through the one or more clinical data points to determine an ideal combination of clinical data points, where the ideal combination of clinical data points are used to improve the probability model created by the prediction system.

The one or more clinical data points are selected from the group consisting of age, biopsy result, digital rectal exam (DRE) result, total prostate specific antigen (PSA) level, free PSA level, number of prior negative biopsies, prostate volume, ethnicity, medication use around the time of biopsy, result of any prostate cancer diagnostic test, or a combination thereof.

In some embodiments, the total PSA level is statistically converted or log transformed before feeding it into the prediction system. In some further embodiments, the free PSA level is statistically converted or transformed to free PSA ratio before feeding it into the prediction system. The free PSA level is converted to free PSA ratio by dividing values of the free PSA level with the total PSA level.

In some embodiments, the free PSA ratio is converted to a free PSA ratio below a linear value, wherein the linear value is the value of free PSA ratio at which the Risk Score starts showing variability. The linear value could be in the range of 0.1-0.5, and the free PSA ratio is converted to the “free PSA ratio below a linear value” using the equation:

$$free PSA ratio below a linear value = max ( linear value - free PSA ratio , 0 )$$

In some preferred embodiments, the free PSA ratio is converted to a free PSA ratio below 0.3, wherein 0.3 is the value of free PSA ratio at which the Risk Score starts showing variability, and using the equation:

$$free PSA ratio below 0.3 = max ( 0 \text{.3} - free PSA ratio , 0 )$$

In some embodiments, the SaMD/CRM or method requires statistical conversion of the age before feeding it into the prediction system, where the age is converted to obtain an age-related risk of grade group ≥2 prostate cancer.

In some alternative embodiments, the SaMD/CRM or method requires that one or more clinical data points or the one or more statistically converted clinical data points are standardized to obtain a standardized feature values (Z-scores) for each clinical data point before feeding it into the prediction system, using the equation:

$$Standardized feature value = \frac{clinical data point value - clinical data point mean value}{clinical data point standard deviation value}$$

In some embodiments, the SaMD/CRM or method further comprises the step of determining diagnostic ability of the prediction system by plotting an Area Under the Curve (AUC) Receiver Operating Characteristic (ROC) curve, where the AUC ROC value may fall in the range of 0.70 to 0.90.

In some embodiments, the prediction system is a computer, an operating system, a software, or a machine learning algorithm where the software module that trains the prediction system to create the probability model is a Tensorflow module or a Python's scikit-learn module. The software module that trains the prediction system to create the neural network model is the Tensorflow module. The software module that trains the prediction system to create the logistic regression model comprises of a Python scikit-learn library software module, Python pandas software module, Python numpy software module, Python onnx software module, Python onnxruntime software module, Python onnxmltools software module, and Python onnxconverter_common software module.

In the embodiments where the Tensorflow module is required, the module uses the following parameters: Neurons in network: 1, learning rate: 0.005, Optimizer: Stochastic gradient descent, Momentum: 0.9, Epochs: 2000, and Batch size: Number of observations in dataset.

In the embodiments where Python's scikit-learn module is required, the module trains the prediction system system by optimizing a set of model hyperparameters to obtain a favorable AOC ROC value using the one or more clinical data points, wherein the model hyperparameters are optimized by grid searching through the model hyperparameters individually and/or in combination. The model hyperparameters are usually selected from Penalty value, tol, C value or solver, where the Penalty value is 11 or 12; where the tol value is 1e−4, where the C value is 0.1, 0.25, 0.5, 1.0, 2.5, 5, 10, 25, 50, 100, 250, 500, or 1000; and where the solver value is liblinear. The Python's onnxmltools software module converts the probability model of the prediction system to ONNX format.

In an alternate embodiment, a kit for predicting a possibility of developing clinically significant prostate cancer is provided that comprises a dataset of one or more clinical data points, means for statistically converting the one or more data points, a prediction system configured to create a probability model using the one or more clinical data points or the statistically converted data points; and a software module configured to train the prediction system to create the probability model using the one or more clinical data points or the statistically converted data points.

In the embodiment recited above, the one or more clinical data points are selected from the group consisting of age, biopsy result, digital rectal exam (DRE) result, total prostate specific antigen (PSA) level, free PSA level, number of prior negative biopsies, prostate volume, ethnicity, medication use around the time of biopsy, result of any prostate cancer diagnostic test, or a combination thereof, and the prediction system is a computer, an operating system, a software, or a machine learning algorithm. Additionally, the software module that trains the prediction system to create the probability model is a Tensorflow module or a Python's scikit-learn module. The software module that trains the prediction system to create the neural network model is the Tensorflow module. The software module that trains the prediction system to create the logistic regression model is the Python's scikit-learn module which comprises a Python's scikit-learn library software module, Python's pandas software module, Python's numpy software module, Python's onnx software module, Python's onnxruntime software module, Python's onnxmltools software module, and Python's onnxconverter_common software module.

In an alternate embodiment a kit for predicting a possibility of developing clinically significant prostate cancer is provided which comprises the software as a medical device or the non-transitory computer readable medium as defined in any one of above embodiments and instructions for performing the method for predicting the possibility of developing clinically significant prostate cancer according to any one of the above embodiments.′

In another embodiment, a non-transitory computer readable medium having stored thereon software instructions that, when executed by a processor, cause the processor to perform the method for predicting the possibility of developing clinically significant prostate cancer according to any one of the above embodiments is provided. A system comprising the SaMD/CRM for predicting a possibility of developing clinically significant prostate cancer according to any of the above embodiments is also provided.

In another embodiment, a software as a medical device for predicting the possibility of developing clinically significant prostate cancer is provided. The software is trained using any of the training methods available in the art to perform the method steps according to any one of the above embodiments.

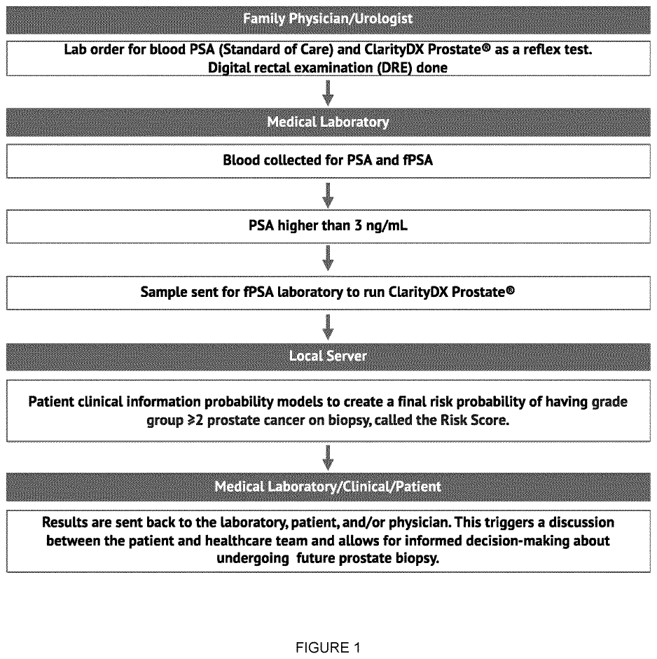

Referring now to FIG. 1, a flow chart is provided to explain how the SaMD/CRM-based probability model would potentially work. After a high PSA test result, a doctor can prescribe SaMD/CRM/ClarityDX Prostate as a reflex text. The SaMD/CRM probability model can predict the possibility of the patient developing or not developing clinically relevant prostate cancer, which can significantly reduce the over diagnosis, and potential overtreatment, of indolent or clinically insignificant prostate cancers.

As shown in the figure, after a high PSA level and DRE, the patient samples are sent for fPSA analysis, and patient characteristics such as age and number of previous negative biopsies, and the results of PSA, DRE and fPSA are fed into the SaMD/CRM prediction system to create probability models that provide final risk probability of having high grade prostate cancer i.e. grade 2 or higher. The results of the probability model i.e. high risk of developing clinically significant prostate cancer or low risk of developing clinically significant prostate cancer; are sent to the laboratory for further discussion between the doctor and the patient. This allows for an informed decision making process.

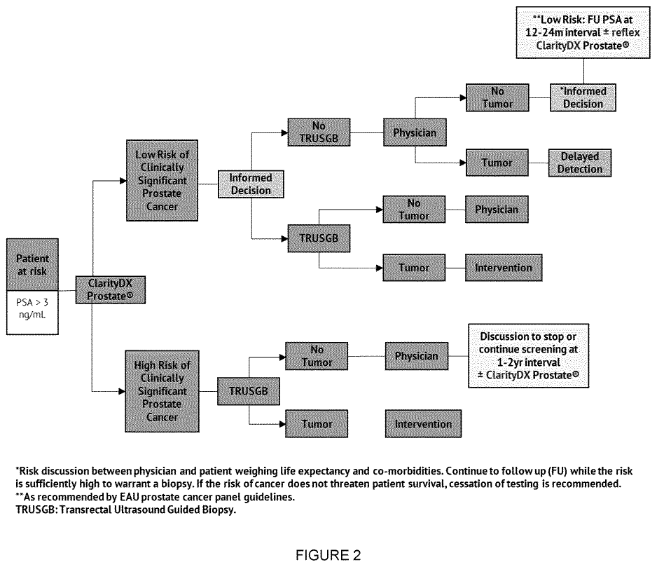

FIG. 2 shows a risk discussion flow chart between a patient who is assessed to have a low risk of clinically significant prostate cancer or a high risk of developing a clinically significant prostate cancer using the SaMD/CRM probability model and his/her doctor. If the probability model assesses a low risk, the patient and the doctor may decide to take a Transrectal Ultrasound Guided Biopsy (TRUSGB) in future or decide against it. Optionally, the doctor may recommend a future physician visit to check for tumors to make an informed decision. If high risk of prostate cancer is predicted, the patient can take TRUSGB once prescribed by the doctor. If TRUSGB identifies a tumor, further intervention may be required. However, if no tumor is detected, the physician and the patient can discuss to stop or continue with the screening every year or two, with or without ClarityDX (SaMD/CRM) analysis. The risk discussion between physician and patients can weigh in on life expectancy and co-morbidities. A follow-up would be recommended if the risk is sufficiently high to warrant a biopsy, however, if the risk of cancer does not threaten patient survival, cessation of testing may be recommended.

The prostate cancer risk stratification test such as SaMD/CRM offers clinicians and patients a chance to clarify the patients' risk of clinically significant prostate cancer before biopsy, and thereby allows for better informed decisions regarding future treatments and interventions. Proof of concept and ClarityDX Prostate® test clinical validation studies with SaMD/CRM, on both prospective and real-world patient samples show that using it as a reflex test after a high PSA test would significantly reduce the number of unnecessary biopsies. This would clear the healthcare system so that only men with clinically significant and potentially life-threatening prostate cancer need to pursue biopsies and treatments. Therefore, using risk calculator stratification tools such as the SaMD/CRM as reflex tests, are predicted to be more cost-effective for the healthcare system and patients and their families.

### Clinical Studies

The Alberta Prostate Cancer Research Initiative (APCaRI) is a province-wide multidisciplinary research program founded in 2012, and their mandate is to develop, validate and translate novel tools to improve prostate cancer detection and to accurately determine the aggressiveness of prostate cancer.

For clinical validation of the ClarityDX (SaMD/CRM), large numbers of high-quality patient samples linked to accurate and complete clinical outcome data was required. APCaRI created the Alberta Prostate Cancer Registry and Biorepository, which collects health information and biofluid samples, respectively, from a prospective cohort of men without a prior diagnosis of prostate cancer who were presented with PSA-screened or clinical suspicion of prostate cancer requiring prostate biopsies or transurethral prostate surgery. The clinical outcomes of the consenting study participants who also donated biospecimen were followed prospectively, with attention to prostate cancer related events including diagnosis, treatment(s), relapse(s) and death.

The studies used machine learning with PSA and fPSA plus clinical information to create a Risk Score. The accuracy of prediction of the various test models is depicted with their resulting Area Under the receiver operating characteristic Curve (AUC) values.

Results of a 1,437 patient APCaRI Cohort

This study used serum samples from an APCaRI cohort of 1,437 patients (1,036 patients in the training group, 401 patients in the validation group). The predictive accuracy of the machine learning probability model combined with the patients' PSA and fPSA levels was compared to competing models and PSA levels alone (PSA).

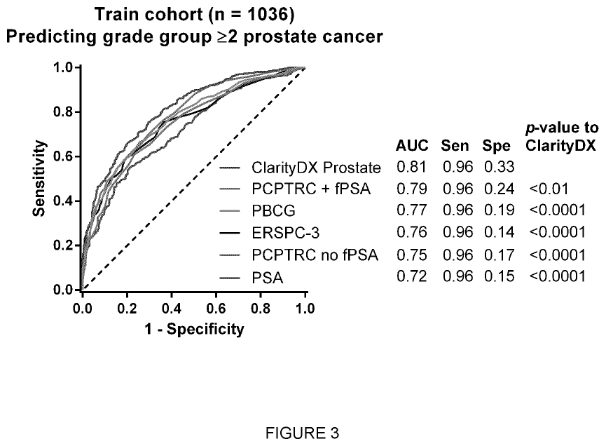

The ClarityDX and SaMD/CRM probability model could predict the biopsies in the validation cohort resulting in benign disease or indolent prostate cancer from grade group ≥2 prostate cancer with an AUC of 0.81 versus, Prostate Cancer Prevention Trial Risk Calculator (PCPTRC) with fPSA (0.77), PCPTRC without fPSA (0.72), ERSPC-3 (0.72) and PSA only (AUC 0.66), as shown in FIG. 3. FIG. 3 shows the Proof of Concept Test AUCs as conducted using an initial 1,437 patient cohort.

### Data Analysis

Machine learning can analyze large, complex disease datasets and rapidly generate disease prediction models to provide accurate patient risk scores; thereby increasing the diagnostic sensitivity and specificity of the test. In addition, computational processing is becoming more powerful and cheaper and data storage is more affordable making using machine learning in diagnostic tests advantageous.

The proposed SaMD/CRM model uses machine learning in the form of binary logistic regression to create a probability model called the Risk Score. A threshold on the Risk Score determines if patients are Low-Risk or High-Risk for clinically significant prostate cancer.

Prostate cancer risk calculators (RCs), such as PCPTRC and European Randomized Study of Screening for Prostate Cancer (ERSPC), use logistic regression machine learning to stratify an individual's risk of prostate cancer using patients' clinical information only and tend to be freely accessible online tools. Studies and meta-studies on RCs show that they were moderate to good at predicting any prostate cancer with AUCs from 0.64 to 0.79.

Researchers have recently shown that merging biomarker datasets with RCs, or selected clinical information can result in higher performances than their standalone tests. For example, prostate cancer diagnostic tests such as SelectMDx, 4Kscore, CNI 2nd Opinion, ExoDx Prostate (ExoDx), MyProstateScore (MPS), and PHI combine patient clinical information and biomarkers with machine learning analysis to improve test model performance. It is important to know that not all clinical information results in improvement of test accuracy. For example, the 4Kscore developers found that including prostate volume data with the kallikrein biomarker characteristics did not improve the discrimination of high-grade prostate cancer, even though prostate volume can predict biopsy outcome.

### Analytical Performance

Analytical method validation is the confirmation, by the examination and provision of objective evidence, that the specifications for the intended use of a test is fulfilled. The SaMD/CRM test uses clinical PSA and fPSA results from certified laboratories. The current study used data from DynaLIFE medical labs using a Roche cobas instrument.

The table below assesses the applicability of different performance claims for the ClarityDX Prostate® test.

| | | | Minimum | |
|---|---|---|---|---|
| | | | samples used | |
| | | | for | Minimum |
| Performance | | | performance | Performance |
| Claim | Applicability | Explanation | evaluation | Metric |
| Diagnostic | Yes | The proportion of men with clinically | 181 | Equal or |
| Sensitivity | | significant prostate cancer which | | greater than |
| | | are identified as being high risk for | | PCPTRC and |
| | | clinically significant prostate cancer | | ERSPC risk |
| | | by the ClarityDX Prostate ® test. | | calculator 3 |
| Diagnostic | Yes | The proportion of men without | 181 | Equal or |
| Specificity | | clinically significant prostate cancer | | greater than |
| | | which are identified as being low | | PCPTRC and |
| | | risk for clinically significant prostate | | ERSPC risk |
| | | cancer by the ClarityDX Prostate ® | | calculator 3 |
| | | test. | | |
| Diagnostic | Yes | The proportion of men considered | 181 | Equal or |
| Positive | | high risk for clinically significant | | greater than |
| Predictive | | prostate cancer with the ClarityDX | | PCPTRC and |
| Value | | Prostate ® test which have clinically | | ERSPC risk |
| | | significant prostate cancer. | | calculator 3 |
| Diagnostic | Yes | The proportion of men considered | 181 | Equal or |
| Negative | | low risk for clinically significant | | greater than |
| Predictive | | prostate cancer with the ClarityDX | | PCPTRC and |
| Value | | Prostate ® test which do not have | | ERSPC risk |
| | | clinically significant prostate cancer. | | calculator 3 |

To obtain relevant and accurate results, tests can be conducted on a group of patients shortlisted using the following criteria and the resulting data can be fed in the machine learning algorithm for conducting predictive analyses. The criterion used is listed below:
Have not been previously diagnosed with prostate cancer,
Between 40 and 75 years of age,
PSA ≥3 ng/ml,
Have provided a blood sample≤12 months of a diagnostic prostate biopsy

However, to avoid ambiguous results the clinical information of patients that fall under any of these categories can be excluded:
Results of diagnostic prostate biopsy not available,
PSA values below 3 ng/ml,
Age is outside the eligibility criteria at the time of participant enrollment,
PSA values are unknown.

As seen from FIG. 3, the previously executed proof of concept (POC) study with the ClarityDX Prostate® test from 377 men provided mean test scores of 29.69 and 37.38 for men without and with clinically significant prostate cancer, respectively. Therefore, solely based on the above proof of concept study, if sample size calculations are to be performed with Python's statsmodels module, a minimum sample size of 181 participants is required for 0.80 power and 0.05 alpha, given the test score standard deviation of 26.04.

Probability models for clinically significant prostate cancer were created using samples and clinical data from research institutes. Finalized and fixed models are evaluated on samples and clinical data and the clinical performance of the SaMD/CRM was evaluated using the following metrics:
Diagnostic Sensitivity,
Diagnostic Specificity,
Diagnostic Positive Predictive Value,
Diagnostic Negative Predictive Value.

The evaluation results were compared to the PCPTRC and the ERSPC risk calculator, but the results can be compared with any other risk calculator for clinically significant prostate cancer.

It is pertinent to note, that SaMD/CRM based probability model faces some limitations, because the prostate biopsy results, as currently used for clinically significant prostate cancer, are not perfectly accurate. Biopsy sampling error sometimes cause some individuals with clinically significant prostate cancer to have negative biopsy results due to biopsy needles missing the tumours or aggressive regions of tumours. Prostate cancer grading is further compromised due to disagreement between pathologists grading the same tissues (kappa coefficients of 0.43 to 0.72). Since the clinical data or identifiers being used have significant error, this may prevent the probability model from having an AUC above ~0.85.

To address the issue of inaccuracies in prostate biopsy results, an option is to collect patient surgical pathology results after they have undergone a radical prostatectomy. Test performance on surgical pathology reports may provide a more accurate measure of test accuracy due to the removal of biopsy sampling error since surgical specimens allow pathological analysis of the entire resected prostate and not just biopsy needle samples.

### Software Validation and Data Storage

The SaMD/CRM probability model uses software that was validated in accordance with IEC 62366-1:2015. More details on software validation can be found in NASOP-0027, Software Development and Validation Process for Medical Devices, and NADOC-0004. The Risk Score values for study participants was aggregated into a CSV file for local analysis.

Observations and Unexpected Outcomes: Unexpected outcomes during performance evaluation were recorded in appropriate validation reports. Unexpected outcomes included:
outliers,
instability of sample or reagent signal,
non-reproducibility,
non-correlation of results to the reference or to the diagnostic pattern,
defects or breakdowns,
software errors, or
error signals.

The errors or deviations were tracked and the cause of errors or deviations was traced whenever possible. Where the validity of previous tests was questionable due to an identified source of error, the tests were repeated after addressing the cause. Unexpected issues caused by misuse or misinterpretation of instructions were noted separately.

### Hardware and Software Used for Analysis

The system specifications for the prediction system used for the analysis of clinical performance are listed below.

| Hardware / Software | Specification / Version |
|---|---|
| CPU | Intel Core i7-6700K |
| Motherboard | Z170-A |
| Windows 10 | 10.0.19043 |
| Python | 3.8 |
| Scikit-learn (Python package) | 0.24.1 |
| Numpy (Python package) | 1.22.3 |
| Pandas (Python package) | 1.2.2 |
| Onnxruntime (Python package) | 1.11.0 |

### Experimental Data

A copy of clinical data from the APCaRI-05 REDCap database was obtained for data analysis. The number of participants with biopsy pathology data, PSA, and age was 1020 from Edmonton, 403 from Calgary, and 29 from John Hopkins University. Not all APCaRI-05 participants had their samples analyzed with the ClarityDX Prostate® test owing to the issues listed in the following table.

| | Number of |
|---|---|
| Issue | participants affected |
| Participant recruitment issue (PSA too | 7 |
| low/high) | |
| Nonconforming results | 146 |

Some participant samples were not run due to multiple issues described above. The final number of APCaRI-05 participants with clinical data and ClarityDX Prostate® test data is noted below.

| | Participants with |
|---|---|
| Clinical Center | Clinical data |
| Kipnes Urology Centre | 1007 |
| Prostate Cancer Center | 401 |
| Johns Hopkins University | 29 |

### Clinical Performance Evaluation Results

Only participants with both ClarityDX Prostate® test and clinical data, including biopsy pathology data, PSA, and age, were used for creating and evaluating the final ClarityDX Prostate® test probability models. Participants from Kipnes Urology Center (Edmonton) and Johns Hopkin University (Baltimore) were grouped into the training cohort for probability model creation (n=1036) and the participants from the Prostate Cancer Center (Calgary) were in the validation cohort for evaluating the final and fixed ClarityDX Prostate® test probability models (n=401).

Below are the clinical results of the probability model for the train cohort using a 18.6% threshold in the ClarityDX Prostate® test for separating participants into Low-Risk and High-Risk groups. Other clinical features and the PCPTRC and ERSPC-3 had thresholds chosen to match as close as possible the sensitivity of the ClarityDX Prostate® test.

| | GG ≤ 1 | GG ≥ 2 | | ROC | | Sensitivity, % | Specificity, % | PPV, % | NPV, % |
|---|---|---|---|---|---|---|---|---|---|
| | PCa | PCa | p-value | AUC (CI) | Cutoff | (CI) | (CI) | (CI) | (CI) |
| Train cohort | | | | | | | | | |
| Patients, n | 558 | 478 | | | | | | | |
| | (54%) | (46%) | | | | | | | |
| Number | 0.14 | 0.040 | <0.0001 | 0.54 | <0.5000 | 96 | 11 | 48 | 78 |
| of prior | (0.42) | (0.22) | | (0.45-0.48) | | (94-98) | (8.4-13) | (45-51) | (67-86) |
| negative | | | | | | | | | |
| biopsies, | | | | | | | | | |
| mean (SD) | | | | | | | | | |
| DRE, n | 74 | 164 | <0.0001 | 0.67 | | 55 | 79 | 69 | 68 |
| (% abnormal) | (13%) | (34%) | | (0.64-0.71) | | (49-61) | (75-83) | (63-74) | (63-72) |
| Age, yr, | 62 | 65 | <0.0001 | 0.65 | >53.50 | 96 | 12 | 48 | 78 |
| median (IQR) | (57-66) | (61-69) | | (0.61-0.68) | | (94-97) | (9.8-15) | (45-52) | (68-85) |
| PSA, ng/ml, | 6.5 | 9.2 | <0.0001 | 0.72 | >4.595 | 96 | 15 | 49 | 80 |
| median (IQR) | (5.0-8.3) | (6.7-15) | | (0.69-0.75) | | (93-97) | (13-19) | (46-52) | (72-87) |
| Free | 1.1 | 1.1 | 0.69 | 0.51 | >0.4465 | 96 | 3.8 | 46 | 50 |
| PSA, ng/ml, | (0.76-1.5) | (0.72-1.7) | | (0.47-0.55) | | (93-97) | (2.3-5.8) | (43-49) | (33-65) |
| median (IQR) | | | | | | | | | |
| Free PSA | 0.16 | 0.11 | <0.0001 | 0.74 | <0.2547 | 96 | 11 | 48 | 74 |
| ratio, | (0.13-0.21) | (0.077-0.15) | | (0.23-0.30) | | (93-97) | (8.1-13) | (45-51) | (62-83) |
| median (IQR) | | | | | | | | | |
| ERSPC-3, | 8.0 | 25 | <0.0001 | 0.76 | >3.500 | 96 | 14 | 49 | 82 |
| median (IQR) | (5.0-15) | (11-44) | | (0.73-0.78) | | (95-98) | (12-17) | (46-52) | (74-89) |
| PCPTRC no | 9.0 | 15 | <0.0001 | 0.75 | >5.500 | 96 | 17 | 50 | 83 |
| free PSA, | (6.0-12) | (10-23) | | (0.72-0.77) | | (94-98) | (14-20) | (46-53) | (75-89) |
| median (IQR) | | | | | | | | | |
| PCPTRC with | 9.0 | 22 | <0.0001 | 0.79 | >5.500 | 96 | 24 | 52 | 88 |
| free PSA, | (6.0-15) | (12-36) | | (0.76-0.81) | | (94-98) | (21-28) | (49-55) | (82-93) |
| median (IQR) | | | | | | | | | |
| PBCG, | 30 | 49 | <0.0001 | 0.77 | >18.50 | 96 | 19 | 50 | 84 |
| median (IQR) | (20-41) | (35-67) | | (0.74-0.80) | | (93-97) | (16-23) | (47-54) | (77-90) |
| ClarityDX | 29 | 63 | <0.0001 | 0.81 | >18.60 | 96 | 33 | 55 | 90 |
| Prostate, | (16-48) | (43-83) | | (0.78-0.83) | | (93-97) | (29-37) | (51-58) | (85-93) |
| median (IQR) | | | | | | | | | |
| Validation cohort | | | | | | | | | |
| Patients, n | 244 | 157 | | | | | | | |
| | (61%) | (39%) | | | | | | | |
| Number | 0.10 | 0.019 | 0.0040 | 0.54 | <0.5000 | 98 | 9.0 | 41 | 88 |
| of prior | (0.34) | (0.14) | | (0.44-0.48) | | (95-99) | (5.8-13) | (36-46) | (68-97) |
| negative | | | | | | | | | |
| biopsies, | | | | | | | | | |
| mean (SD) | | | | | | | | | |
| DRE, n | 54 | 75 | <0.0001 | 0.63 | | 53 | 72 | 58 | 68 |
| (% abnormal) | (22%) | (48%) | | (0.57-0.68) | | (45-61) | (65-78) | (49-67) | (61-74) |
| Age, yr, | 62 | 66 | <0.0001 | 0.64 | >52.50 | 94 | 9.0 | 40 | 69 |
| median (CI) | (57-66) | (60-69) | | (0.59-0.70) | | (89-97) | (5.8-13) | (35-45) | (50-84) |
| PSA, ng/ml, | 6.5 | 8.4 | <0.0001 | 0.66 | >4.595 | 94 | 15 | 41 | 78 |
| median (CI) | (5.1-8.5) | (6.5-11) | | (0.61-0.71) | | (89-97) | (11-20) | (36-46) | (64-88) |
| Free | 1.3 | 1.1 | 1.00 | 0.53 | <3.295 | 94 | 3.7 | 38 | 50 |
| PSA, ng/ml, | (0.88-1.8) | (0.84-1.8) | | (0.41-0.54) | | (89-97) | (1.8-7.1) | (33-43) | (21-72) |
| median (CI) | | | | | | | | | |
| Free PSA | 0.19 | 0.13 | <0.0001 | 0.72 | <0.2308 | 94 | 29 | 45 | 89 |
| ratio, | (0.14-0.24) | (0.10-0.18) | | (0.23-0.34) | | (89-97) | (24-36) | (39-51) | (79-95) |
| median (CI) | | | | | | | | | |
| ERSPC3, | 8.0 | 22 | <0.0001 | 0.72 | >5.500 | 90 | 31 | 45 | 82 |
| median (CI) | (5.0-18) | (9.0-40) | | (0.67-0.77) | | (84-94) | (25-37) | (40-51) | (73-89) |
| PCPTRC no | 9.0 | 14 | <0.0001 | 0.72 | >6.500 | 93 | 24 | 44 | 84 |
| free PSA, | (7.0-13) | (10-19) | | (0.66-0.76) | | (88-96) | (19-30) | (39-50) | (74-92) |
| median (CI) | | | | | | | | | |
| PCPTRC with | 7.0 | 15 | <0.0001 | 0.77 | >5.500 | 94 | 32 | 47 | 90 |
| free PSA, | (5.0-12) | (10-25) | | (0.72-0.82) | | (90-97) | (27-38) | (42-53) | (82-95) |
| median (CI) | | | | | | | | | |
| PBCG, | 30 | 50 | <0.0001 | 0.74 | >20.50 | 94 | 24 | 44 | 87 |
| median (CI) | (22-42) | (33-61) | | (0.68-0.78) | | (90-97) | (19-30) | (39-50) | (76-93) |
| ClarityDX | 25 | 58 | <0.0001 | 0.81 | >18.60 | 94 | 37 | 49 | 90 |
| Prostate, | (15-44) | (42-76) | | (0.76-0.85) | | (89-97) | (31-43) | (43-54) | (82-95) |
| median (CI) | | | | | | | | | |

GG: Grade group
PCa: Prostate cancer
ROC AUC: Receiver operating characteristic area under the curve
CI: Confidence interval (95%)
PPV: Positive predictive value
NPV: Negative predictive value
SD: Standard deviation
DRE: Digital rectal exam
IQR: Interquartile range
PSA: Prostate-specific antigen
ERSPC-3: European Randomized study of Screening for Prostate Cancer risk calculator 3
PCPTRC: Prostate Cancer Prevention Trial Risk Calculator 2.0
PBCG: Prostate Biopsy Collaborative Group risk calculator

Using only the validation cohort for evaluation, as seen from the table above, the SaMD/CRM probability model met or exceeded all clinical performance metrics as shown in the table below.

| | Minimum | | Actual |
|---|---|---|---|
| | Performance | | Performance |
| Clinical Performance Claim | Metric | | Metric |
| Diagnostic Sensitivity | ≥ERSPC-3 | (90%) | 94% |
| | ≥PCPTRC | (93%) | |
| Diagnostic Specificity | ≥ERSPC-3 | (31%) | 37% |
| | ≥PCPTRC | (24%) | |
| Diagnostic Positive Predictive | ≥ERSPC-3 | (45%) | 49% |
| Value | ≥PCPTRC | (44%) | |
| Diagnostic Negative Predictive | ≥ERSPC-3 | (82%) | 90% |
| Value | ≥PCPTRC | (84%) | |

| | | |
|---|---|---|
| Clinical Reproducibility | ≤10% | 0.43% |

### SaMD/CRM Model Training and Test Datasets Description

The logistic regression model used in the SaMD/CRM test was trained on anonymized data from the APCaRI-05 clinical study including 1007 men from Edmonton and 29 men from John Hopkins University (1036 men total). Features used for training the logistic regression model include:
Age (years),
Total PSA (ng/ml),
Free PSA (ng/mL),
DRE (0=normal; 1=abnormal), and
Number of prior negative prostate biopsies (e.g., 0, 1, 2)

The trained logistic regression model was fixed and evaluated on the test dataset which included 401 men from Calgary.

### Machine Learning Training Process

### 1. Logistic Regression Model

System performing model training: Model training was executed on a Windows 10 Pro desktop system using Python's scikit-learn library for model creation. The hardware and software specifications used are detailed below.

Hardware

| Item | Value |
|---|---|
| Processor | Intel Core i7-6700K |
| Motherboard | ASUS Z170-A |
| Total Physical RAM | 64 GB |
| Storage drive | Corsair Force LE SSD (960 GB) |

Software

| Item | Value/version |
|---|---|
| Operating system | Microsoft Windows 10 Pro |
| Python | 3.8.2 |
| scikit-learn (Python module) | 1.1.1 |
| pandas (Python module) | 1.4.2 |
| numpy (Python module) | 1.22.4 |
| onnx (Python module) | 1.11.0 |
| onnxruntime (Python module) | 1.11.0 |
| onnxmltools (Python module) | 1.11.0 |
| onnxconverter_common (Python module) | 1.9.0 |

Feature imputation: Missing values for the following identifiers:
free PSA,
number of prior negative biopsies, and
digital rectal exam findings
   were imputed with the median training data values. Full data was available for PSA and age in the train and test datasets.

Feature preprocessing: It is pertinent to note that PSA values (ng/ml) were log transformed before training. Apart from that, free PSA ratio was calculated as shown below using original (non-log transformed) PSA data:

$$free PSA ratio = free PSA ( ng / mL ) / total PSA ( ng / mL )$$

Model Training: Logistic regression models were trained using scikit-learn's LogisticRegression class. Model hyperparameters were optimized with the training cohort by grid searching through all combinations of the hyperparameters found in the table below. Model hyperparameters that provided the highest receiver operating characteristic area under the curve (ROC AUC) value in the training data when using 5 repeats of 5-fold cross-validation were used during model training on the entire training data for final model creation.

The table below lists the logistic regression hyperparameters tested on the training dataset:

| Hyperparameter | Hyperparameter values |
|---|---|
| Penalty | l1, l2 |
| C | 0.1, 0.25, 0.5, 1, 2.5, 5, 10, 25, 50, 100, 250, |
| | 500, 1000 |

All logistic regression models were optimized by grid searching all of the combinations of hyperparameters above. Upon completion of model training, the onnxmltools module was used to convert the logistic regression model into ONNX format. Advantages of using ONNX models include:
Faster model inference,
Ability to perform model inference on any system with an ONNX runtime library which includes most major platforms (Windows, macOS, Linux, Android, IOS) and programming languages (Python, C++, C#, C, Java, and JavaScript).

### Model Training Results

It was observed that log-transforming total PSA improved logistic regression model performance on the test dataset compared to using the original total PSA.

| | Test ROC AUC |
|---|---|
| total PSA | 0.803 |
| log total PSA | 0.808 |

Additionally, transforming free PSA into free PSA ratio improved logistic regression model performance on the test dataset as shown below.

| | Test ROC AUC |
|---|---|
| free PSA | 0.754 |
| free PSA ratio | 0.808 |

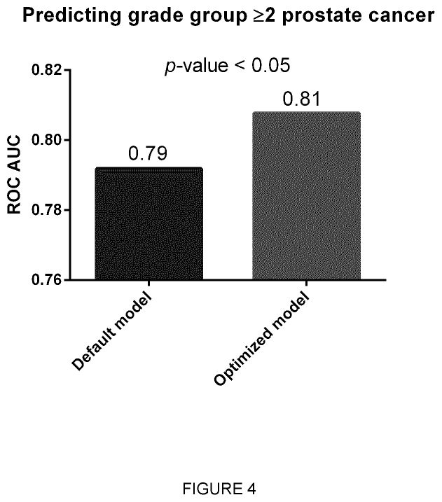

Grid search through the logistic regression hyperparameters and finding the ideal parameters on the training dataset also improved logistic regression model performance on the test dataset compared to using default parameters. FIG. 4 shows a comparison chart of ROC AUC values of the default and optimized models.

There was a total of 153 observations and unexpected outcomes as described above which prevented samples from these participants from being analyzed. The final number of study participants in the training cohort was 1036 while 401 participants were used in the validation cohort for evaluating the ClarityDX Prostate® test and probability model.

Using a probability threshold of 18.6%, the ClarityDX Prostate® test had a greater sensitivity, specificity, positive predictive value, and negative predictive value than the PCPTRC risk calculator by 1%, 13%, 5%, and 6%, respectively. Using the same threshold, ClarityDX Prostate® test had a greater sensitivity, specificity, positive predictive value, and negative predictive value than the ERSPC-3 risk calculator by 4%, 6%, 4%, and 8%, respectively.

From the results presented above, it is clear that clinical performance of the ClarityDX Prostate® test based probability model is superior to the ERSPC risk calculator 3, suggesting that the ClarityDX Prostate® (SaMD/CRM) test has clinical value in better informing doctors of the probability of finding clinically significant prostate cancer, defined as grade group 2 or greater, in prostate biopsy which may improve the selection of which patients receive prostate biopsies to decrease the number of unnecessary biopsies.

### 2. Neural Network Model

Similar to the logistic regression analysis, the neural network model used in the ClarityDX Prostate® test was trained on anonymized data from the APCaRI-05 clinical study including 1007 men from the Kipnes Urology Center in Edmonton and 29 men from John Hopkins University (1036 men total). Raw features used for training the neural network model included:
Age (years),
Total PSA (ng/ml),
Free PSA (ng/ml),
Digital Rectal Exam findings (DRE) (0=normal; 1=abnormal), and
Number of prior negative prostate biopsies (e.g., 0, 1, 2)

Some of the features such as Total PSA, free PSA, and/or age were modified to obtain more accurate results. The trained neural network model was fixed and evaluated on the test dataset which included 401 men from the Prostate Cancer Center in Calgary.

Feature Preprocessing: The Total PSA values (ng/ml) obtained from the test dataset were log transformed before training the Clarity DX neural network model. The Free PSA levels were transformed to obtain a Free PSA ratio. The ratio was calculated as shown below using original (non-log transformed) PSA data:

$$free PSA ratio = free PSA ( ng / mL ) / total PDA ( ng / mL )$$

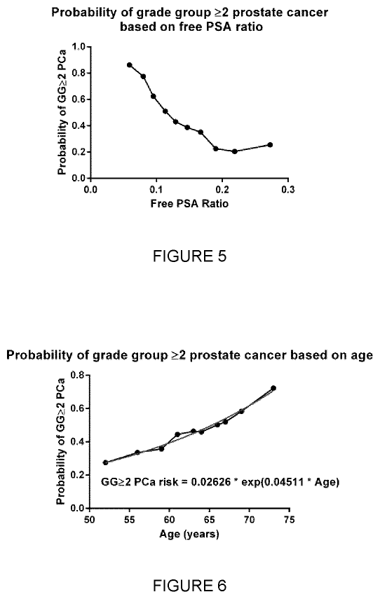

From the training data, it was found that the risk of grade group ≥2 prostate cancer was not linear for all possible free PSA ratios since the risk slope decreased substantially by free PSA ratio of 0.2, as shown in FIG. 5. The probability of grade group 2 prostate cancer based on free PSA ratio is shown in FIG. 5.

In an exemplary embodiment, a new model feature was created to normalize the results and further improve the accuracy of the neural network model, when the amount of free PSA ratio was below a linear value. The linear value was determined based on the dataset and the value at which the risk slope starts showing some variability. It's the value of free PSA ratio at which the Risk Score starts showing variability. The free PSA ratio is then converted to obtain a free PSA ratio below a linear value using the equation:

$$free PSA ratio below a linear value = max ( linear value - free PSA ratio , 0 )$$

The linear value can be anywhere in the range of 0.1-0.5. In the above-referenced dataset, the linear value was found to be 0.3 i.e. the variability started at 0.3. Only positive values were accepted thus a free PSA ratio above 0.3 was given a value of 0. The following equation was used to modify the values:

$$free PSA ratio below 0.3 = max ( 0 \text{.3} - free PSA ratio , 0 )$$

A person skilled in the art would understand that the value of the Free PSA ratio i.e. the linear value, can be anywhere between 0.1-0.5 depending on the dataset.

The table below provides examples of how different free PSA ratios would be converted into free PSA ratio below 0.3:

| Free PSA ratio | Free PSA ratio below 0.3 |
|---|---|
| 0.01 | 0.29 |
| 0.1 | 0.2 |
| 0.2 | 0.1 |
| 0.3 | 0 |
| 0.4 | 0 |

As a result, the feature “free PSA ratio below 0.3” was used as input into the neural network instead of the actual free PSA ratio.

Additionally, the age of the patients was not directly employed in the neural network model since the risk of grade group ≥2 prostate cancer was not linearly related to age as shown in FIG. 6. Using the training data, the risk of grade group ≥2 prostate cancer was fit to the exponential growth equation shown in FIG. 6. For each patient needing a ClarityDX Prostate/SAMD/CRM test result, the patient's age was used in the equation in FIG. 6 to determine the age-related risk of grade group ≥2 prostate cancer and this age-related risk was used as an input into the neural network instead of years of age. FIG. 6 shows the probability of grade group 2 prostate cancer based on age of the patient.

Feature Standardization: Before training the neural network Clarity DX model, all input features, including:
Log PSA,
Free PSA ratio below 0.3
Age-related GG≥2 PCa risk,
Number of previous negative biopsies,
Digital rectal exam findings,
were standardized by converting their values into standardized feature values (Z-scores) using the equation below.

$$Standardized feature value = \frac{clinical data point value - clinical data point mean value}{clinical data point standard deviation value}$$

The clinical data point mean and standard deviation values required for the above equation were calculated from all available data from the training dataset. The standardized feature values (Z-scores) represent the number of standard deviations the feature is from the mean.

Feature imputation: In the event of missing values for any input feature, the values were imputed with the median value from the training dataset.

Neural network models were trained using Tensorflow using the parameters below:
Neurons in network: 1,
learning rate: 0.005,
Optimizer: Stochastic gradient descent,
Momentum: 0.9
Epochs: 2000
Batch size: 1036 (can change depending on the size of training data)
   A single neuron could potentially behave as logistic regression and could replace the neural network.

In some embodiments, the logistic regression models were trained using scikit-learn using the parameters below:
penalty: 12,
tol: 1e−4,
C: 1.0,
solver: liblinear
   No grid searching of hyperparameters occurred in these models to reduce the amount of variability. Although, in these models, the feature preprocessing steps used for the neural network model were employed.

As can be seen from the following tables, the feature modifications or rather pre-processing steps improves the neural network and the logistic regression models performance of the test dataset. Log-transforming total PSA improves neural network and logistic regression models performance on the test dataset compared to using the original total PSA.

Test ROC AUC

| Feature | Logistic regression | Neural network |
|---|---|---|
| total PSA | 0.743 | 0.723 |
| log total PSA | 0.756 | 0.750 |

Transforming free PSA into “free PSA ratio below 0.3” improves both models performance on the test dataset.

Test ROC AUC

| | Logistic | |
|---|---|---|
| Feature | regression | Neural network |
| Free PSA | 0.743 | 0.723 |
| Free PSA ratio below 0.3 | 0.801 | 0.794 |

Transforming age into age-related grade group ≥2 prostate cancer risk slightly improves neural network and logistic regression models performance on the test dataset.

Test ROC AUC

| | Logistic | |
|---|---|---|
| Feature | regression | Neural network |
| Age (years) | 0.743 | 0.723 |
| Age-related GG ≥ 2 PCa risk | 0.745 | 0.726 |

If all the feature preprocessing methods described above are used, it substantially improves the model performance as is evident from the results provided in the table below.

Test ROC AUC

| | Logistic | |
|---|---|---|
| Feature | regression | Neural network |
| PSA | 0.743 | 0.723 |
| Free PSA | | |
| Age (years) | | |
| Number of negative biopsies | | |
| DRE | | |
| Log PSA | 0.812 | 0.812 |
| Free PSA ratio below 0.3 | | |
| Age-related GG ≥ 2 PCa risk | | |
| Number of negative biopsies | | |
| DRE | | |

It is pertinent to note that feature standardization is important for creating accurate models since removing feature standardization decreases clinical performance of the model as is evident from the table below.

| | |
|---|---|
| Model training method | Test ROC AUC |

| (Using all feature preprocessing | Logistic | |
|---|---|---|
| methods described above) | regression | Neural network |
| Without feature standardization | 0.792 | 0.752 |
| With feature standardization | 0.812 | 0.812 |

### Embodiments Pertaining to the Second Computer Implemented Method, System and CRM

In another aspect of the invention, the invention describes a method for predicting risk, probability or possibility of developing clinically significant prostate cancer. The method could be used as a preventive diagnostic tool to avert or avoid unnecessary biopsies that are based on a few patient characteristics or feature e.g. PSA results.

In the method, one or more clinical data points, features or patient characteristics are obtained from a plurality of patients. The patients are selected based on a set of pre-determined criteria. The clinical data points, features or characteristics could be obtained from a subset of the population or from a variety of sources. These features or characteristics may vary based on the region, geography, age and other contributory factors. Some of the data points, features or characteristics may be processed, engineered or standardized before employing them to predict the risk of prostate cancer.

One or more model hyper parameters are optimized by a grid searching technique. The model hyper parameters could be any statistical formula or standardization technique that would help improve or optimize the analysis of the one or more clinical data points or optimize the risk prediction capability of the proposed technique. The model hyperparameters is usually a parameter whose value is used to control or adjust the learning process. In an embodiment of the invention, the model hyper parameters could be penalty, C, solver, tol, n_neighbors, degree, splitter, min_samples_split, min_samples_leaf, criterion, max_depth, n_estimators, learning_rate, num_leaves, hidden_layer_sizes, activation, learning_rate_init alpham and/or solver. A person skilled in the art would readily understand that the above hyper parameters could vary considerably and may have a range of values.

The one or more clinical data points, features or characteristics are fed into a predicting system. In an embodiment of the invention, some or all of the data points are processed before they are fed into the prediction system. One or more optimized model hyper parameters are also fed into the prediction system along with the clinical data points. The method further involves training the prediction system using a machine learning or software module to generate a risk score based on the processed clinical data points and the model hyper parameters. The model hyper parameters control the learning process. The prediction system's performance may be optimized by feeding in clinical data points from various sources.

The machine learning or software module trains the prediction system by optimizing the model hyperparameters to obtain a favorable AOC ROC value using the one or more clinical data points, wherein the model hyperparameters are optimized by grid searching through the model hyperparameters individually and/or in combination.

In the method, the prediction system predicts the risk of developing clinically significant prostate cancer based on the generated risk score. In an embodiment of the invention, when a given patient's data, features or characteristics are fed into the prediction system, the trained prediction system generates a risk score using the data points and hyper parameters that were used to train the prediction system. Based on the risk score, the prediction system may recommend that the patient seeks immediate clinical evaluation, biopsy and/or medical intervention if the risk score is high or recommend future clinical evaluation if the risk score is low.

In some embodiments, the set of pre-determined criteria for selecting the plurality of patients could be any patient feature or characteristic that may be related or have any significance on the prostate cancer diagnosis or prognosis. In some embodiments, the method involves selecting patients based on patient age, prior prostate cancer diagnosis, referral for biopsy, total prostate specific antigen (total PSA) result, prostate volume or MRI prostate volume, digital rectal exam result, PI-RADS score or a combination of any the above.

In some embodiments, the step of processing one or more clinical data points, features or characteristics may be carried out by scaling the one or more clinical data points by calculating a z-score. By way of scaling the clinical data points may be standardized to obtain a standardized feature value (Z-score) for each clinical data point before feeding it into the prediction system, the standardized feature value is obtained using an equation:

$$Standardized feature value = \frac{clinical data point value - clinical data point mean value}{clinical data point standard deviation value}$$

In some embodiments, the step of processing the one or more clinical data points, features or characteristics may be carried out by performing median feature value imputation on the one or more clinical data points. In some other embodiments, the processing step may be performer by statistically converting the one or more clinical data points. In some other embodiments, the processing step is carried out by determining a threshold value for the one or more clinical data points by calculating percentage sensitivity for clinically significant prostate cancer for each clinical data point. In some alternate embodiments, the data points may be processed by determining confidence intervals for the one or more clinical data points by bias-correction and accelerated bootstrapping. In an alternate embodiment, the clinical data points may be processed by performing a decision curve analysis on the one or more clinical data points. The processing step may also be carried out by determining statistical significance of the one more clinical data points by calculating p-values≤0.05. The clinical features and data could be fed into the prediction system without processing, or by modifying the data points using one or a combination of processing steps note above.

The one or more clinical data points, features or characteristics that may be fed into the prediction system could be total PSA (in ng/ml), free PSA (in ng/ml), % free PSA (calculated using the formula 100*free PSA/total PSA), age (in years), ethnicity features like Caucasian (yes or no), African American (yes or no), Asian (yes or no), Native American (yes or no), Latin (yes or no), digital rectal exam (DRE) findings (normal or abnormal), family history of prostate cancer, previous negative biopsy (yes or no), or the number of previous negative biopsies.

In the processing step, the one or more clinical data points may be statistically converted by log-transforming the one or more clinical data points. For instance, total PSA may be statistically converted to obtain the log PSA value and the converted log PSA value is fed into the prediction system. In some embodiments, free PSA may be transformed using the following formula before feeding its value in the prediction system:

$$\% F ree PSA ratio = 100 * free PSA ( ng / mL ) / total PSA ( ng / mL ) .$$

In some embodiments, the one or more clinical data points may be statistically converted by standardizing the value of one or more clinical data points or one or more statistically converted clinical data points. In some other embodiments, the one or more clinical data points may be statistically converted by engineering the one or more clinical data points using quantile grouping. In quantile grouping the one or more clinical data points are plotted in each quantile against the probability of developing clinically significant prostate cancer. In some embodiments, the one or more clinical data points are further engineered by creating a random forest model with a subset of the one or more clinical data points and further calibrating the model with isotonic regression. For instance, patient age could be engineered to obtain a modified patient age related risk feature to improve the prediction system's performance.

In some of the embodiments, the one or more data points (for e.g. total PSA level) is log-transformed before feeding it into the prediction system. For instance, the free PSA level may be statistically converted before feeding it into the prediction system. In some embodiments, the patient age is converted to obtain an age-related risk of grade group ≥2 prostate cancer before feeding it into the prediction system.

In some embodiments, the model hyper parameters are optimized by grid searching through various combinations of the model hyper parameters to determine a receiver operating characteristic area under the curve (ROC AUC) value. The hyper parameters are optimized to obtain a higher ROC AUC value to improve the prediction system's outcome. The ROC AUC value can be determined by using a five-fold cross-validation technique. In some embodiments, the training step may involve training the prediction system to predict the risk of developing clinically significant prostate cancer using previous biopsy results. The training set may use previous biopsy results from various sources and ethnic groups.

In some embodiments, the method may further comprise the step of grid searching through the one or more clinical data points to determine an ideal combination of clinical data points. The ideal combination of clinical data points could then be used to improve the probability model created by the prediction system.

Any known prediction system known in the art could be employed. However, in some embodiments, the prediction system is logistic regression, linear discriminant analysis, quadratic discriminant analysis, k-nearest neighbors, linear basis function support vector machines, radial basis function support vector machines, single decision tree, random forest, lightGBM, XGBoost, or multilayer perceptron. In some embodiments, the prediction system is multilayer perceptron or random forest. In some embodiments, the system which provides the higher ROC AUC value is used. In some embodiments a combination of prediction systems could be employed. In some other embodiments, the prediction system is a computer, an operating system, a software, or a machine learning algorithm.

Various machine learning modules known in the art can be employed to train the prediction systems note above. A few non-limiting examples could be Python, LightGBM or XGBoost.

In some embodiments, the method involves comparing the risk score or one of the accuracy metrics of the prediction system with a previous biopsy result or with a score generated by another risk calculator or with the accuracy metrics of another third party risk calculator known in the art to ascertain accuracy or specificity of the prediction system. For instance, the accuracy of the prediction system may be ascertained by determining diagnostic specificity, diagnostic sensitivity, diagnostic positive predictive value, diagnostic ability, diagnostic negative predictive value, or using a combination of any one of the above. The diagnostic specificity of the prediction system could be determined by using a risk score threshold of >25%. The diagnostic ability of the prediction system may be determined by plotting a plurality of ROC AUC curves. Any accuracy metric for instance diagnostic ability or AUC values of the prediction system can be compared with the diagnostic ability or AUC values of another risk calculator to determine accuracy of the prediction system.

In some embodiments, the method involves a step of improving the performance of the prediction system by using different subsets of the training data and calibrating the model or the prediction system with isotonic regression. For instance, different random forest models could be created using different subsets of training data and the different versions are then calibrated using isotonic regression.

In some embodiments, the method involves a step of providing a recommendation based on the risk score generated by the prediction system. For instance, if the risk score is high based on the risk score threshold of >25% the prediction system may recommend immediate clinical evaluation, biopsy and/or medical intervention; and if the risk score is low based on the risk score threshold of ≥25% the system may recommend future clinical evaluation or other preventive measures if needed.

In another embodiment of the invention, a non-transitory computer readable medium having stored thereon an algorithm that, when executed by a processor, cause the processor to perform the method for predicting the risk of developing clinically significant prostate cancer, wherein the algorithm is trained to perform the method steps noted above.

In some alternate embodiments, a kit for predicting risk of developing clinically significant prostate cancer is also provided. The kit comprises a non-transitory computer readable medium and instructions for performing the method for predicting the risk of developing clinically significant prostate cancer. The instructions could also be for training the computer readable medium to predict the risk of developing prostate cancer.

In some other embodiment, a software as a medical device is provided. The software is capable of predicting the risk of developing clinically significant prostate cancer, wherein the software is trained to perform the method steps noted hereinbefore. The software may be trained using one of the machine learning modules or alternate techniques known in the art.

In some alternate embodiments, a system that comprises the above-recited non-transitory computer readable medium for predicting the risk of developing clinically significant prostate cancer is also envisioned.

In some embodiments, the above system may be used to predict the risk of developing clinically significant prostate cancer, or to avoid biopsy for diagnosing the risk of developing clinically significant prostate cancer, or as an early diagnostic tool to determine the risk of developing clinically significant prostate cancer, or to identify patients with a higher risk of developing clinically significant prostate cancer.

Early prostate cancer diagnosis guideline documents recommend using PSA plus another test, such as a biomarker test, risk predictor model, or multi-parametric magnetic resonance imaging (mpMRI) to improve the sensitivity and specificity for clinically significant prostate cancer before biopsy.

In the present invention, the inventors have aimed to create improved risk models predicting any prostate cancer and clinically significant prostate cancer, defined as GG ≥2, using PSA, free PSA, and other useful clinical features. Accurate risk models predicting prostate biopsy results would improve the selection of men requiring MRI and prostate biopsies, lowering healthcare costs, and minimizing patient adverse events. A wide range of conventional and advanced machine learning models were examined and compared using cohorts from three clinical sites. The optimized models, called ClarityDX Prostate, demonstrated state-of-the-art results for predicting biopsy results in the pre-MRI setting. ClarityDX Prostate is simpler, faster, and more accessible than MRI, thereby making it a highly feasible approach to improve patient-clinician decision-making about whether to biopsy or to perform more expensive diagnostic procedures.

### Experimental Data: Materials and Methods

Without wishing to be bound by theory or experimental results, the following paragraphs describe the nature of the invention by way of examples only. The experiments, or specific examples of materials, and formulations described should not be construed as limiting the scope of the invention. A person skilled in the art would readily understand and appreciate that other materials and/or methods not specifically described also form part of the invention.

The Alberta Prostate Cancer Research Initiative (APCaRI) is a province-wide multidisciplinary research program founded in 2012, and their mandate is to develop, validate and translate novel tools to improve prostate cancer detection and to accurately determine the aggressiveness of prostate cancer.

The APCaRI-05 study data was used to create the ClarityDX Prostate test which predicts prostate cancer and grade group (GG) ≥2 prostate cancer. APCaRI-05 participants, recruited from three different clinical sites, included 1409 men which were between 40 and 75 years of age, had total PSA results ≥3 ng/ml, and were referred for prostate biopsies. Risk models to predict prostate cancer (all grade groups) or GG ≥2 prostate cancer were fit using data from 967 participants from the University of Alberta (Edmonton, Canada) and Johns Hopkins University (Maryland, US) while fixed models were validated on data from 442 participants from the University of Calgary (Calgary, Canada). A variety of machine learning models were evaluated, and neural networks were used to create the final ClarityDX Prostate models which used data from PSA, free PSA, age, prior negative biopsy status, digital rectal exam findings, ethnicity, and family history of prostate cancer as model features. ClarityDX Prostate was compared to PSA, ERSPC-3, PCPTRC, and PBCG risk calculators for predicting prostate cancer and grade group (GG) ≥2 prostate cancer.

### Study Design

Data from the APCaRI-05 study was collected from five centers including the University of California, Los Angeles (Los Angeles, USA), University of Calgary (Calgary, Canada), Johns Hopkins University (Baltimore, USA), University of Alberta (Edmonton, Canada), and Thomayer University Hospital (Prague, Czechia). Patient data was collected and enrollment criteria included: 1) males between 40-75 (inclusive) years of age, 2) no prior prostate cancer diagnosis and who are referred to have a prostate biopsy, and 3) total PSA results ≥3 ng/ml collected within six months of enrollment. Exclusion criteria included a prior diagnosis of cancer, excluding non-melanoma skin cancer.

Of the 1583 patients in the APCaRI-05 REDCap database, 32 patients were removed due to not meeting protocol eligibility (e.g., PSA below 3 ng/ml, having a prior cancer, etc), and 142 patients were removed due to not having required analysis features which included age, biopsy results, PSA, and free PSA. Risk models to predict prostate cancer (all grade groups) or GG ≥2 prostate cancer were derived using data from patients recruited from the University of Alberta and Johns Hopkins University (n=967). After derivation, the models were fixed and subsequently validated on a separate cohort of patients recruited from the University of Calgary (n=442).

All patients in the cohort gave written informed consent and the study was approved by the Health Research Ethics Board of the recruitment sites under the APCaRI-01 protocol (HREBA-CC-18-0513). The study methodologies conformed to the standards set by the Declaration of Helsink. The ClarityDX Prostate test results were not provided to the clinical sites for patient care, and the laboratory personnel who performed the tests were blinded for patient characteristics.

### PSA and Free PSA Tests

For patients recruited from the University of Calgary and University of Alberta, upon patient enrollment, serum samples were collected, and processed as described previously³⁴; aliquots frozen at −80° C. were shipped to DynaLIFE Medical Labs (Edmonton, Canada) for batch analysis of total PSA and free PSA using a Roche Cobas 8000 e801 system. Total PSA and free PSA results were acquired within the 12-week stability range for free PSA and were not subjected to multiple freeze-thaw cycles. For the other clinical sites, PSA and free PSA results were acquired as part of standard clinical practice and provided for analysis in this study.

### Prostate Biopsies

Prostate biopsies were performed according to standard practice for the clinical sites which predominantly included transrectal ultrasound-guided 12-core biopsies. Gleason grading followed previously described methods for the University of Alberta and University of Calgary.

### Predictive Models

Predictive model input features were processed before model training. Feature scaling was performed by calculating z-scores. The scaling was performed for input features for logistic regression, linear and quadratic discriminant analysis, K-nearest neighbors, linear and radial basis function support vector machines, and multiplayer perceptron models. Median feature value imputation (determined from the train cohort) was used for all input features except for LightGBM and XGBoost models, which can intrinsically handle missing data.

Eleven different machine learning models were compared, including logistic regression, linear and quadratic discriminant analysis, k-nearest neighbors, linear and radial basis function support vector machines, single decision tree, random forest, lightGBM, XGBoost, and multilayer perceptron. Models were trained to predict GG ≥2 prostate cancer from biopsy results.

ClarityDX Prostate models used modified features such as log PSA as well as age-related risk of GG ≥2 prostate cancer. The age-related risk feature was created by plotting the probability of GG ≥2 prostate cancer over age where patient ages were grouped into 5 quintiles. Within each quintile, the median age and fraction of patients with GG ≥2 prostate cancer were used for plotting. Data in plots were fitted using linear regression or an exponential growth curve using GraphPad Prism 9.5.1 software. The exponential growth equations were used to determine each patient's age-related risk of GG ≥2 prostate cancer by inputting patient age and solving for risk of GG ≥2 prostate cancer which was used in model training.

All predictive models were trained using Python 3.8 with scikit-learn (1.1.2) except LightGBM, XGBoost, and neural network models which used the lightgbm (3.3.5) and xgboost (1.7.5) packages, respectively. Model hyperparameters were optimized with the train cohort by grid searching through all combinations of the hyperparameters found in Table 1. Table 1 provides model hyperparameters tested during model optimization. Model hyperparameters that provided the highest receiver operating characteristic area under the curve (ROC AUC) value in the train cohort when using 5 repeats of 5-fold cross-validation were used during model training on the entire training data for final model creation. Models created from the train cohort were fixed and used for inference on the validation cohort for cross-clinical site model evaluation. Predictive models were compared to the PBCG risk calculator predictions which were obtained using an R script.

TABLE 1

| | Hyperparameter values |
|---|---|
| Logistic regression | |
| Penalty | l1, l2 |
| C | 0.1, 0.25, 0.5, 1, 2.5, 5, 10, 25, 50, 100, 250, |
| | 500, 1000 |
| Linear discriminant | |
| analysis | |
| Solver | svd, lsqr, eigen |
| Tol | 0.00001, 0.0001, 0.001 |
| Quadratic discriminant | |
| analysis | |
| Tol | 0.00001, 0.0001, 0.001 |
| K-Nearest Neighbors | |
| n_neighbors | 1, 3, 5, 10, 15, 20, 30, 40, 50, 60, 70, 80, 90, |
| | 100 |
| Linear support | |
| vector machines | |
| C | 0.1, 0.25, 0.5, 1, 2.5, 5, 10, 25, 50, 100, 250, |
| | 500, 1000 |
| Tol | 0.0001, 0.001, 0.03 |
| Radial basis function | |
| support vector machines | |
| C | 0.1, 0.25, 0.5, 1, 2.5, 5, 10, 25, 50, 100, 250, |
| | 500, 1000 |
| Degree | 2, 3 |
| Decision tree | |
| Splitter | best, random |
| min_samples_split | 1, 2, 3, 4, 5, 10, 15, 20, 25 |
| min_samples_leaf | 1, 2, 3, 4, 5, 10, 15, 20, 25 |
| Random Forest | |
| Criterion | gini, entropy |
| max_depth | 3, 4, 5, 6, 7 |
| n_estimators | 20, 50, 100, 200, 400, 600 |
| LightGMB | |
| learning_rate | 0.01, 0.03, 0.1 |
| num_leaves | 3, 5, 10, 30, 100 |
| n_estimators | 50, 100, 200 |
| XGBoost | |
| n_estimators | 50, 100, 200, 400 |
| max_depth | 1, 2, 3, 4 |
| learning_rate | 0.01, 0.03, 0.1 |
| Multilayer Perceptron | |
| hidden_layer_sizes | 1, 2, 3, 4, 5 |
| Activation | identity, logistic, tanh, relu |
| learning_rate_init | 0.3, 0.01, 0.003, 0.001, 0.0003, 0.0001 |
| alpha | 0.00001, 0.0001, 0.001 |
| solver | sgd, lbfgs, adam |

### Statistical Analyses

All statistics and model calibration curve values were calculated using Python 3.8 software with scikit-learn (1.1.2), scipy (1.9.2), and scikits.bootstrap (1.1.0) packages. When comparing 2 groups, Mann-Whitney U-tests were used for continuous data and Fisher's exact tests were used for binary categorical data. Statistical significance was determined by p-values≤0.05. Clinical features that were analyzed included total prostate specific antigen (PSA, ng/ml), free PSA (ng/ml), % free PSA (100*free PSA/total PSA), age (years), Caucasian (yes or no), African American (yes or no), Asian (yes or no), Native American (yes or no), Latin (yes or no), digital rectal exam (DRE) findings (normal or abnormal), family history of prostate cancer, previous negative biopsy (yes or no), and the number of previous negative biopsies. Where DRE results were available from both a general practitioner physician as well as a urologist, the urologist DRE results were used. ROC curves were compared by DeLong's method. ClarityDX Prostate threshold values for the train cohort were determined using approximately 95% sensitivity for GG ≥2 prostate cancer. Threshold values for other clinical features and PBCG predictions were set to match the sensitivity of ClarityDX Prostate when possible or select a threshold providing a lower sensitivity when it was not possible to match sensitivities. Confidence intervals were determined with 10,000 resamples of bias-corrected and accelerated bootstrapping. Decision curve analysis was performed using Python 3.9 and the dcurves (1.0.6.2) library.

### Results

Several clinical features differed between clinical sites as shown in Table 2. While all sites recruited predominantly Caucasian participants, Johns Hopkins University had the highest proportion of African Americans at 19% while University of Calgary had the lowest at 1.4%. Prior negative biopsies were less than 8% from the University of Alberta and the University of Calgary while the Thomayer University Hospital had the most with 50% of participants with a prior negative biopsy. Median PSA was lowest from Thomayer University Hospital and Johns Hopkins University at 6.2 ng/ml and highest at the University of Alberta at 7.4 ng/ml. Biopsy results to be predicted in this study showed Thomayer University Hospital having the highest proportion of negative biopsies at 50% while Johns Hopkins University had the lowest at 27%. Age and % free PSA were relatively similar between sites with median values between 63-65 years and 14-16%, respectively. The proportion of patients with GG ≥2 prostate cancer was relatively similar between clinical sites with Johns Hopkins University having the highest at 47% while Thomayer University Hospital having the lowest at 37%. Although relatively few University of Alberta participants had pre-biopsy MRIs, the detection rate of GG ≥2 prostate cancer was not increased through the use of pre-biopsy MRI (4/15 (27%) GG ≥2 prostate with pre-biopsy MRI vs 169/427 (40%) GG ≥2 prostate without pre-biopsy MRI; p-value=0.42).

Table 2 provides patient characteristics by clinical site.

| | Training | | | | | | Validation | | | |
|---|---|---|---|---|---|---|---|---|---|---|
| | University | | | | | | | | | |
| | of | | | | Johns | | | | Thomayer | |
| | California, | | University | | Hopkins | | University | | University | |
| | Los Angeles | | of Calgary | | University | | of Alberta | | Hospital | |
| Patients, n | 1394 | | 442 | | 355 | | 938 | | 319 | |
| Age, yr, median (IQR) | 65 | (59-70) | 64 | (58-68) | 65 | (60-70) | 63 | (58-68) | 64 | (57-68) |
| Ethnicity, n (%) | | | | | | | | | N/A | |
| Caucasian | 935 | (67%) | 376 | (85%) | 263 | (74%) | 745 | (79%) | | |
| African American | 71 | (5.1%) | 6 | (1.4%) | 66 | (19%) | 38 | (4.1%) | | |
| Asian | 85 | (6.1%) | 48 | (11%) | 8 | (2.3%) | 88 | (9.4%) | | |
| Native American | 1 | (0.072%) | 1 | (0.23%) | 0 | (0%) | 34 | (3.6%) | | |
| Latin | 8 | (0.57%) | 6 | (1.4%) | 7 | (2.0%) | 17 | (1.8%) | | |
| Other | 63 | (4.5%) | 5 | (1.1%) | 6 | (1.7%) | 15 | (1.6%) | | |
| Unknown | 231 | (17%) | 0 | (0.0%) | 5 | (1.4%) | 1 | (0.11%) | | |
| Family history PCa, n (% | | | | | N/A | | | | N/A | |
| abnormal) | | | | | | | | | | |
| Yes | 341 | (24%) | 118 | (27%) | | | 290 | (31%) | | |
| No | 1014 | (73%) | 301 | (68%) | | | 585 | (62%) | | |
| Unknown | 39 | (2.8%) | 23 | (5.2%) | | | 63 | (6.7%) | | |
| Previous negative | | | | | | | | | | |
| biopsy, n (%) | | | | | | | | | | |
| Yes | 518 | (37%) | 34 | (7.7%) | 59 | (17%) | 65 | (6.9%) | 161 | (50%) |
| No | 876 | (63%) | 408 | (92%) | 296 | (83%) | 872 | (93%) | 158 | (50%) |
| Unknown | 0 | (0%) | 0 | (0%) | 0 | (0%) | 1 | (0.11%) | 0 | (0%) |
| Number of previous | 0.50 | (0.73) | 0.090 | (0.34) | 0.22 | (0.60) | 0.083 | (0.32) | 0.96 | (1.2) |
| negative biopsies, mean | | | | | | | | | | |
| (SD) | | | | | | | | | | |
| DRE, n (% abnormal) | | | | | | | | | | |
| Yes | 187 | (13%) | 144 | (33%) | 70 | (20%) | 225 | (24%) | 25 | (7.8%) |
| No | 835 | (60%) | 249 | (56%) | 264 | (74%) | 367 | (39%) | 294 | (92%) |
| Unknown | 372 | (27%) | 49 | (11%) | 21 | (5.9%) | 346 | (37%) | 0 | (0%) |
| PSA, ng/ml, median | 6.6 | (4.8-9.4) | 7.3 | (5.6-9.9) | 6.2 | (4.7-8.4) | 7.4 | (5.6-11) | 6.2 | (4.2-9.4) |
| (IQR) | | | | | | | | | | |
| Free PSA, ng/ml, median | 0.98 | (0.66-1.5) | 1.2 | (0.85-1.8) | 0.90 | (0.60-1.4) | 1.1 | (0.74-1.5) | 0.88 | (0.57-1.5) |
| (IQR) | | | | | | | | | | |
| % Free PSA, median | 15 | (10-20) | 16 | (11-21) | 14 | (10-20) | 14 | (9.5-19) | 15 | (11-19) |
| (IQR) | | | | | | | | | | |
| Biopsy results, n (%) | | | | | | | | | | |
| Negative | 561 | (40%) | 142 | (32%) | 95 | (27%) | 275 | (29%) | 158 | (50%) |
| Grade Group 1 | 253 | (18%) | 127 | (29%) | 92 | (26%) | 224 | (24%) | 45 | (14%) |
| Grade Group 2 | 266 | (19%) | 125 | (28%) | 78 | (22%) | 291 | (31%) | 50 | (16%) |
| Grade Group 3 | 118 | (8.5%) | 30 | (6.8%) | 36 | (10%) | 107 | (11%) | 26 | (8.2%) |
| Grade Group 4 | 84 | (6.0%) | 7 | (1.6%) | 15 | (4.2%) | 10 | (1.1%) | 23 | (7.2%) |
| Grade Group 5 | 112 | (8.0%) | 11 | (2.5%) | 39 | (11%) | 31 | (3.3%) | 17 | (5.3%) |

Clinical features were individually assessed for predicting GG ≥2 prostate cancer in the training cohort as shown in Table 3. Clinical features that significantly differed between patients without and with GG≥2 prostate cancer in the training cohort included previous negative biopsy (34% vs 20%, p-value<0.0001), number of previous negative biopsies (mean 0.45 vs 0.26, p-value<0.0001), abnormal DRE (11% vs 29%, p-value<0.0001), age (median 63 years vs 66 years, p-value<0.0001), PSA (median 6.2 ng/mL vs 7.7 ng/ml, p-value<0.0001), and % free PSA (17% vs 12%, p-value<0.0001). PSA and % free PSA were the most predictive single features for GG ≥2 prostate cancer with ROC AUC values of 0.63 and 0.69, respectively.

TABLE 3
Predictive value of single features in the training cohort for GG ≥ 2 prostate cancer.

| | Predicting grade group ≥2 prostate cancer | | | | | |
|---|---|---|---|---|---|---|
| | GG ≤ 1 PCa | | GG ≥ 2 PCa | | p-value | ROC AUC (CI) |
| Patients, n | 1270 | (58%) | 921 | (42%) | | |
| Caucasian, n (%) | 923 | (73%) | 651 | (71%) | 0.29 | 0.51 (0.49-0.53) |
| African American, n (%) | 31 | (2.4%) | 35 | (3.8%) | 0.076 | 0.51 (0.50-0.51) |
| Asian, n (%) | 93 | (7.3%) | 48 | (5.2%) | 0.052 | 0.51 (0.50-0.52) |
| Native American, n (%) | 1 | (0.079%) | 1 | (0.11%) | 1.0 | 0.50 (0.50-0.50) |
| Latin, n (%) | 12 | (0.94%) | 9 | (0.98%) | 1.0 | 0.50 (0.50-0.50) |
| Family history PCa, n (%) | 281 | (22%) | 178 | (19%) | 0.29 | 0.51 (0.49-0.53) |
| Prior negative biopsy, n (%) | 428 | (34%) | 183 | (20%) | <0.0001 | 0.57 (0.55-0.59) |
| Number of prior negative | 0.45 | (0.73) | 0.26 | (0.56) | <0.0001 | 0.57 (0.55-0.59) |
| biopsies, mean (SD) | | | | | | |
| DRE, n (% abnormal) | 136 | (11%) | 265 | (29%) | <0.0001 | 0.58 (0.56-0.60) |
| Age, yr, median (IQR) | 63 | (58-68) | 66 | (61-71) | <0.0001 | 0.60 (0.58-0.63) |
| PSA, ng/ml, median (IQR) | 6.2 | (4.6-8.5) | 7.7 | (5.5-11) | <0.0001 | 0.63 (0.61-0.65) |
| Free PSA, ng/ml, median (IQR) | 1.0 | (0.70-1.5) | 0.95 | (0.66-1.5) | 0.19 | 0.52 (0.49-0.54) |
| % Free PSA, median (IQR) | 17 | (13-22) | 12 | (9.0-17) | <0.0001 | 0.69 (0.67-0.71) |

GG: Grade group
PCa: Prostate cancer
ROC AUC: Receiver operating characteristic area under the curve
CI: Confidence interval (95%)
SD: Standard deviation
DRE: Digital rectal exam
IQR: Interquartile range
PSA: Prostate-specific antigen

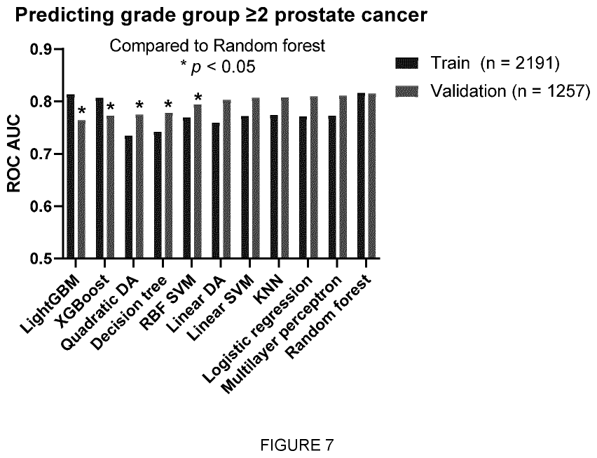

Eleven different machine learning algorithms were compared for predicting GG ≥2 prostate cancer as shown in FIG. 7. FIG. 7 shows a comparison of eleven machine learning algorithms for predicting grade group ≥2 prostate cancer using the receiver operating characteristic area under the curve (ROC AUC) values. ROC AUC values were compared using DeLong's method. The following initialisms were used: GBM: gradient-boosting machine, DA: discriminant analysis, RBF: radial basis function, SVM: support vector machines, KNN: k-nearest neighbors.

Five features were used for models predicting GG ≥2 prostate cancer including PSA, % free PSA, age, digital rectal exam findings (DRE), and previous negative biopsy status (yes or no). The three models with the highest AUC values (0.81) for the validation cohort include random forest, multilayer perceptron, and logistic regression. Other ensemble decision tree algorithms, including LightGBM and XGBoost models, overfit the training cohort data since they had high AUCs for the train cohort (≥0.8) but the lowest AUCs for the validation cohort (<0.78). The random forest model was capable of AUCs greater than 0.8 for both the train and validation cohorts. It was found that model parameter optimization worked well for clinical performance since not performing this optimization did occasionally reduce the random forest model AUC value to 0.78 for the validation cohort (data not shown).

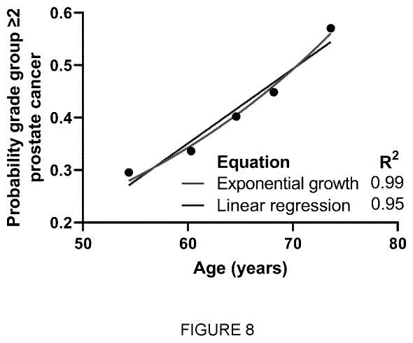

Additional feature engineering was performed on age since it exhibited a non-linear relationship with the probability of GG ≥2 prostate cancer. When plotting patient age and prostate cancer probability in the train cohort, the data fitted an exponential growth equation better than linear regression (R squared 0.99 vs. 0.95, as shown in FIG. 8. FIG. 8 shows the correlation between age and grade group ≥2 prostate cancer probability in the train cohort. An exponential growth equation has a higher R squared value than linear regression when fitting age and probability of grade group ≥2 prostate cancer.

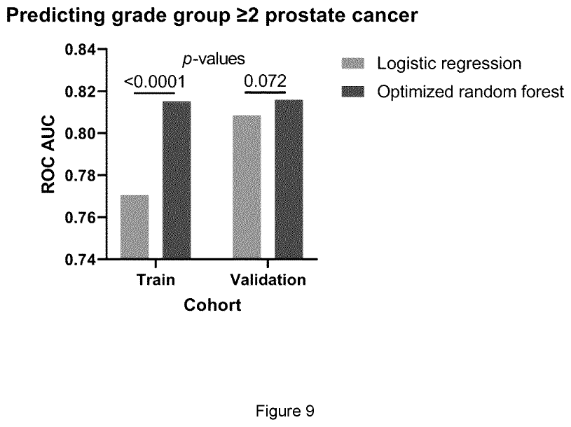

The age-related GG ≥2 prostate cancer feature used the exponential growth curve to determine the risk of GG ≥2 prostate cancer which was used as an input feature for predictive models. To further improve model performance, 50 different random forest models were created with different subsets of the training data and the models were calibrated with isotonic regression. The optimized random forest model had higher AUC values than logistic regression with the largest improvement in the train cohort (0.82 vs 0.77, p-value <0.0001, as shown in FIG. 9. FIG. 9 shows optimized neural network model has improved receiver operating characteristic area under the curve (ROC AUC) values over default logistic regression models for predicting grade group ≥2 prostate cancer in training and validation cohorts. Receiver characteristic curve area under the curve (ROC AUC) values were compared using DeLong's method.

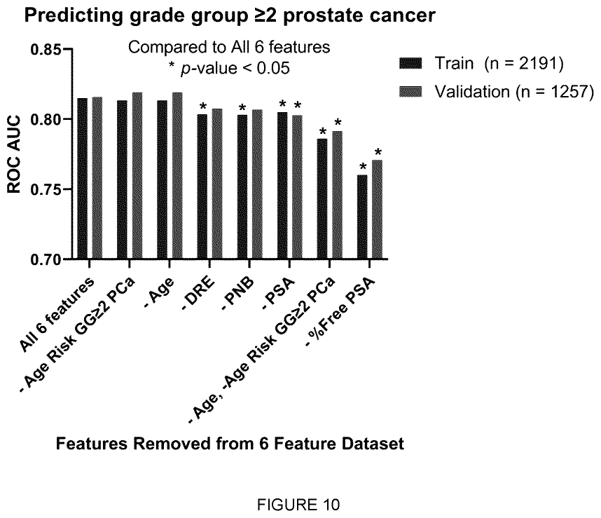

Feature elimination from the optimized random forest model was used to determine the importance of each feature for predicting GG ≥2 prostate cancer, as shown in FIG. 10. FIG. 10 shows Feature selection by feature elimination for models predicting grade group ≥2 prostate cancer. Free PSA ratio was the most important feature for all models since its removal caused the largest decrease in model receiver operating characteristic area under the curve (ROC AUC). ROC AUC values were compared to the inclusion of all features using DeLong's method. GG≥2 PCa, grade group ≥2 prostate cancer, DRE: digital rectal exam findings (normal or abnormal), PNB: prior negative biopsies status (yes or no), PSA: prostate specific antigen.

Model AUC decreased the most when removing % free PSA with AUC values decreasing by 0.055 and 0.045 for the train and validation cohorts (p-value <0.05), respectively. PSA was the next most important feature since removing PSA decreased AUC values by 0.01 and 0.013 in the train and validation cohorts, respectively (p-value <0.05). Individually removing age or the age-related risk of GG ≥2 PCa had non-significant AUC value changes for the train and validation cohort. Removing both age features significantly decreased the AUC for both cohorts below 0.792 (p-value <0.05). Both age-based features were kept in the final model since using both features better balanced model AUC between the train and validation cohorts. Individually removing digital rectal exam findings and prior negative biopsy features decreased AUC values for both cohorts by at least 0.008 although the decrease was only significant in the train cohort.

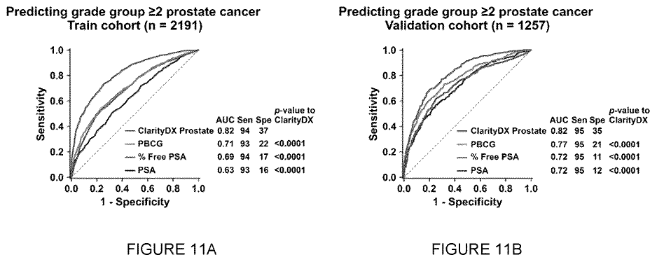

The final optimized calibrated random forest ensemble model was tested further and the model prediction for GG ≥2 prostate cancer was called the Risk Score. When compared to PSA, % free PSA, and PBCG, ClarityDX Prostate provided the highest AUC value for predicting GG ≥2 prostate cancer for the train and validation cohorts, as shown in FIGS. 11A and 11B, with the following ranking by AUC on the validation cohort: ClarityDX Prostate (0.82), PBCG (0.77, p-value<0.01), % free PSA (0.72, p-value<0.0001), and PSA (0.72, p-value<0.0001).

FIG. 11 shows receiver operating characteristic curves for ClarityDX Prostate, PBCG, % Free PSA, and PSA when predicting grade group ≥2 prostate cancer in the training cohort (A) and validation cohort (B). Area under the curve (AUC) values were compared using DeLong's method. ClarityDX Prostate had significantly greater than AUC values than all other tested risk calculators and PSA for predicting prostate cancer and grade group ≥2 prostate cancer in the training and validation cohorts. Sen: sensitivity, Spe: specificity.

To calculate ClarityDX Prostate sensitivity and specificity, a Risk Score threshold of 25% was used since it provided ≥94% sensitivity in both the training and validation cohorts (Table 4, and 5). For both the train and validation cohorts, ClarityDX Prostate had a specificity that was ≥14% (absolute value) higher than PBCG (as shown in Table 4) and ≥21% (absolute value) higher than PSA and % free PSA while maintaining a sensitivity equal or greater than the other tests. ClarityDX Prostate AUC values for predicting GG ≥2 prostate cancer were not significantly different for cohorts including or excluding participants with prior negative biopsies with AUC, sensitivity, and specificity values ≥0.81, ≥94%, and ≥35%, respectively, for both the train and validation cohorts, as shown in Table 6.

Table 4 provides comparison of single features, PBCG, and ClarityDX Prostate for predicting grade group ≥2 prostate cancer in the train cohort.

| | GG ≤ 1 | GG ≥ 2 | | ROC | | Sensitivity, % | Specificity, % | PPV, % | NPV, % |
|---|---|---|---|---|---|---|---|---|---|
| | PCa | PCa | p-value | AUC (CI) | Cutoff | (CI) | (CI) | (CI) | (CI) |
| Patients, n | 1270 (58%) | 921 (42%) | | | | | | | |
| Prior | 428 (34%) | 183 (20%) | <0.0001 | 0.57 (0.55-0.59) | ≥0.5 | 80 (77-83) | 34 (31-36) | 47 (44-49) | 70 (66-74) |
| negative | | | | | | | | | |
| biopsy, n (%) | | | | | | | | | |
| DRE, n (% | 136 (11%) | 265 (29%) | <0.0001 | 0.58 (0.56-0.60) | ≥0.5 | 32 (29-35) | 85 (83-87) | 66 (61-71) | 57 (55-60) |
| abnormal) | | | | | | | | | |
| Age, yr, | 63 (58-68) | 66 (61-71) | <0.0001 | 0.60 (0.58-0.63) | ≥54.99 | 93 (91-94) | 13 (11-15) | 43 (41-46) | 70 (64-76) |
| median (IQR) | | | | | | | | | |
| PSA, ng/ml, | 6.2 (4.6-8.5) | 7.7 (5.5-11) | <0.0001 | 0.63 (0.60-0.65) | ≥4.065 | 93 (91-94) | 16 (14-18) | 45 (42-47) | 76 (70-80) |
| median (IQR) | | | | | | | | | |
| % Free | 17 (13-22) | 12 (9-17) | <0.0001 | 0.69 (0.67-0.71) | <0.2484 | 94 (92-95) | 17 (15-19) | 45 (43-47) | 78 (73-83) |
| PSA, | | | | | | | | | |
| median (IQR) | | | | | | | | | |
| PBCG, | 25 (16-35) | 38 (25-54) | <0.0001 | 0.71 (0.68-0.73) | ≥14.41 | 93 (91-94) | 22 (20-24) | 46 (44-48) | 80 (76-84) |
| median (IQR) | | | | | | | | | |
| ClarityDX | 30 (21-44) | 58 (41-79) | <0.0001 | 0.82 (0.80-0.83) | ≥25 | 94 (92-95) | 37 (34-40) | 52 (50-54) | 89 (86-91) |
| Prostate, | | | | | | | | | |
| median (IQR) | | | | | | | | | |

GG: Grade group
PCa: Prostate cancer
ROC AUC: Receiver operating characteristic area under the curve
CI: Confidence interval (95%)
PPV: Positive predictive value
NPV: Negative predictive value
DRE: Digital rectal exam
IQR: Interquartile range
PSA: Prostate-specific antigen
PBCG: Prostate Biopsy Collaborative Group risk calculator

Table 5 provides comparison of single features, PBCG, and ClarityDX Prostate for predicting grade group ≥2 prostate cancer in the validation cohort.

| | GG ≤ 1 | GG ≥ 2 | | ROC | | Sensitivity, % | Specificity, % | PPV, % | NPV, % |
|---|---|---|---|---|---|---|---|---|---|
| | PCa | PCa | p-value | AUC (CI) | Cutoff | (CI) | (CI) | (CI) | (CI) |
| Patients, n | 702 (56%) | 555 (44%) | | | | | | | |
| Prior negative | 158 (23%) | 68 (12%) | <0.0001 | 0.55 (0.53-0.57) | ≥0.5 | 88 (85-90) | 23 (20-26) | 47 (44-50) | 70 (64-76) |
| biopsy, n (%) | | | | | | | | | |
| DRE, n (% | 73 (10%) | 177 (32%) | <0.0001 | 0.66 (0.63-0.69) | ≥0.5 | 46 (40-50) | 86 (83-89) | 71 (65-76) | 68 (64-71) |
| abnormal) | | | | | | | | | |
| Age, yr, | 62 (56-66) | 65 (61-70) | <0.0001 | 0.65 (0.62-0.68) | ≥54.97 | 94 (92-96) | 16 (14-19) | 47 (44-50) | 78 (70-84) |
| median (IQR) | | | | | | | | | |
| PSA, ng/ml, | 6.2 (4.8-8.4) | 9.1 (6.5-15) | <0.0001 | 0.72 (0.69-0.74) | ≥4.0 | 95 (95-97) | 12 (12-15) | 46 (41-48) | 78 (74-82) |
| median (IQR) | | | | | | | | | |
| % Free PSA, | 16 (12-21) | 11 (8-15) | <0.0001 | 0.72 (0.69-0.75) | <25.08 | 95 (92-96) | 11 (8.8-13) | 46 (43-49) | 72 (63-80) |
| median (IQR) | | | | | | | | | |
| PBCG, median | 24 (16-37) | 46 (31-64) | <0.0001 | 0.77 (0.74-0.79) | ≥14.38 | 95 (92-96) | 21 (18-24) | 49 (46-52) | 83 (77-88) |
| (IQR) | | | | | | | | | |
| ClarityDX | 32 (22-47) | 63 (44-82) | <0.0001 | 0.82 (0.79-0.84) | ≥25 | 95 (94-97) | 35 (32-39) | 54 (51-57) | 91 (87-94) |
| Prostate, | | | | | | | | | |
| median (IQR) | | | | | | | | | |

GG: Grade group
PCa: Prostate cancer
ROC AUC: Receiver operating characteristic area under the curve
CI: Confidence interval (95%)
PPV: Positive predictive value
NPV: Negative predictive value
DRE: Digital rectal exam
IQR: Interquartile range
PSA: Prostate-specific antigen
PBCG: Prostate Biopsy Collaborative Group risk calculator

Table 6 provides ClarityDX Prostate clinical performance including and excluding patients with a prior negative biopsy.

| | Patient | ROC | | Speci- | p- |
|---|---|---|---|---|---|
| Cohort | number | AUC | Sensitivity | ficity | value |
| Train including PNB | 2191 | 0.82 | 94 | 37 | 0.78 |
| Train excluding PNB | 1580 | 0.81 | 94 | 36 | |
| Validation including PNB | 1257 | 0.82 | 95 | 35 | 0.91 |
| Validation excluding PNB | 1030 | 0.81 | 95 | 39 | |

ROC AUC: Receiver operating characteristic area under the curve,
PNB: prior negative biopsy

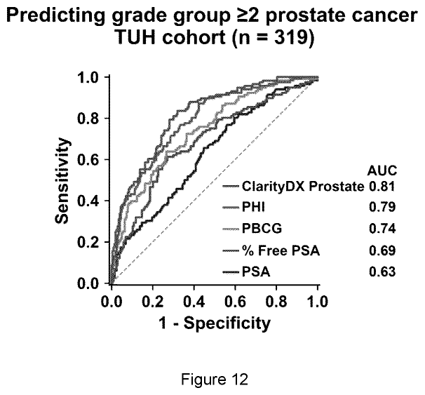

The Thomayer University Hospital had Prostate Health Index (PHI) tests results which were compared to ClarityDX Prostate as shown in FIG. 12. FIG. 12 shows receiver operating characteristic curves for ClarityDX Prostate, PHI, PBCG, % Free PSA, and PSA when predicting grade group ≥2 prostate cancer for Thomayer University Hospital patients.

For predicting GG ≥2 prostate cancer, ClarityDX Prostate had a higher AUC value (0.79 vs 0.82) although they were not significantly different given the relatively smaller cohort size of 355 patients (p-value >0.05).

When assuming a prostate biopsy was performed if the Risk Score was ≥25%, an estimated 35% of unnecessary prostate biopsies could be avoided in the validation cohort while missing 11% GG ≥1, 4.5% GG ≥2, 3.7% GG ≥3, 2.5% GG ≥4, and 2.1% GG 5 prostate cancers, as shown in Table 7. Table 7 shows Prostate cancers found, missed, and biopsies avoided using ClarityDX Prostate model in validation cohort.

TABLE 7

| | GG ≥ 1 | GG ≥ 1 | GG ≥ 2 | GG ≥ 2 | GG ≥ 3 | GG ≥ 3 | GG ≥ 4 | GG ≥ 4 | GG > 5 | GG > 5 | | Unnecessary |
|---|---|---|---|---|---|---|---|---|---|---|---|---|
| | PCa | PCa | PCa | PCa | PCa | PCa | PCa | PCa | PCa | PCa | Biopsies | biopsies |
| | found | missed | found | missed | found | missed | found | missed | found | missed | avoided* | avoided** |
| Thresholds | n (%) | n (%) | n (%) | n (%) | n (%) | n (%) | n (%) | n (%) | n (%) | n (%) | n (%) | n (%) |
| 5 | 823 | 1 | 555 | 0 | 214 | 0 | 81 | 0 | 48 | 0 | 7 | 7 |
| | (99.9%) | (0.1%) | (100%) | (0.0%) | (100%) | (0.0%) | (100%) | (0.0%) | (100%) | (0.0%) | (0.6%) | (1.0%) |
| 10 | 813 | 11 | 555 | 0 | 214 | 0 | 81 | 0 | 48 | 0 | 38 | 38 |
| | (98.7%) | (1.3%) | (100%) | (0.0%) | (100%) | (0.0%) | (100%) | (0.0%) | (100%) | (0.0%) | (3.0%) | (5.4%) |
| 15 | 796 | 28 | 551 | 4 | 213 | 1 | 81 | 0 | 48 | 0 | 98 | 94 |
| | (96.6%) | (3.4%) | (99.3%) | (0.7%) | (99.5%) | (0.5%) | (100%) | (0.0%) | (100%) | (0.0%) | (7.8%) | (13.4%) |
| 20 | 779 | 45 | 545 | 10 | 210 | 4 | 80 | 1 | 47 | 1 | 160 | 150 |
| | (94.5%) | (5.5%) | (98.2%) | (1.8%) | (98.1%) | (1.9%) | (98.8%) | (1.2%) | (97.9%) | (2.1%) | (12.7%) | (21.4%) |
| 25 | 732 | 92 | 530 | 25 | 206 | 8 | 79 | 2 | 47 | 1 | 272 | 247 |
| | (88.8%) | (11.2%) | (95.5%) | (4.5%) | (96.3%) | (3.7%) | (97.5%) | (2.5%) | (97.9%) | (2.1%) | (21.6%) | (35.2%) |
| 30 | 689 | 135 | 517 | 38 | 203 | 11 | 79 | 2 | 47 | 1 | 361 | 323 |
| | (83.6%) | (16.4%) | (93.2%) | (6.8%) | (94.9%) | (5.1%) | (97.5%) | (2.5%) | (97.9%) | (2.1%) | (28.7%) | (46.0%) |
| 35 | 630 | 194 | 480 | 75 | 188 | 26 | 74 | 7 | 44 | 4 | 469 | 394 |
| | (76.5%) | (23.5%) | (86.5%) | (13.5%) | (87.9%) | (12.1%) | (91.4%) | (8.6%) | (91.7%) | (8.3%) | (37.3%) | (56.1%) |
| 40 | 579 | 245 | 448 | 107 | 179 | 35 | 70 | 11 | 40 | 8 | 560 | 453 |
| | (70.3%) | (29.7%) | (80.7%) | (19.3%) | (83.6%) | (16.4%) | (86.4%) | (13.6%) | (83.3%) | (16.7%) | (44.6%) | (64.5%) |
| 45 | 519 | 305 | 409 | 146 | 165 | 49 | 62 | 19 | 37 | 11 | 655 | 509 |
| | (63.0%) | (37.0%) | (73.7%) | (26.3%) | (77.1%) | (22.9%) | (76.5%) | (23.5%) | (77.1%) | (22.9%) | (52.1%) | (72.5%) |
| 50 | 459 | 365 | 378 | 177 | 154 | 60 | 60 | 21 | 36 | 12 | 741 | 564 |
| | (55.7%) | (44.3%) | (68.1%) | (31.9%) | (72.0%) | (28.0%) | (74.1%) | (25.9%) | (75.0%) | (25.0%) | (58.9%) | (80.3%) |

GG: Grade group
PCa: Prostate cancer
*Patients receiving a biopsy but had a Risk Score below the threshold
**Patients with a negative biopsy or grade group 1 PCa with a Risk Score below the threshold

Similar results were observed in the train cohort with a threshold of ≥25% predicting 94% of GG ≥2 prostate cancers and missing 14% GG ≥1, 6.3% GG ≥2, 3.5% GG ≥3, 3.4% GG ≥4, and 2.5% GG 5 prostate cancers, Table 8. Table 8 shows Prostate cancers found, missed, and biopsies avoided using ClarityDX Prostate model in Train cohort.

TABLE 8

| | GG ≥ 1 | GG ≥ 1 | GG ≥ 2 | GG ≥ 2 | GG ≥ 3 | GG ≥ 3 | GG ≥ 4 | GG ≥ 4 | GG > 5 | GG > 5 | | Unnecessary |
|---|---|---|---|---|---|---|---|---|---|---|---|---|
| | PCa | PCa | PCa | PCa | PCa | PCa | PCa | PCa | PCa | PCa | Biopsies | biopsies |
| | found | missed | found | missed | found | missed | found | missed | found | missed | avoided* | avoided** |
| Thresholds | n (%) | n (%) | n (%) | n (%) | n (%) | n (%) | n (%) | n (%) | n (%) | n (%) | n (%) | n (%) |
| 5 | 1381 | 12 | 919 | 2 | 451 | 1 | 267 | 1 | 162 | 0 | 43 | 41 |
| | (99.1%) | (0.9%) | (99.8%) | (0.2%) | (99.8%) | (0.2%) | (99.6%) | (0.4%) | (100%) | (0%) | (2.0%) | (3.2%) |
| 10 | 1368 | 25 | 918 | 3 | 451 | 1 | 267 | 1 | 162 | 0 | 104 | 101 |
| | (98.2%) | (1.8%) | (99.7%) | (0.3%) | (99.8%) | (0.2%) | (99.6%) | (0.4%) | (100%) | (0%) | (4.7%) | (8.0%) |
| 15 | 1339 | 54 | 911 | 10 | 450 | 2 | 266 | 2 | 162 | 0 | 183 | 173 |
| | (96.1%) | (3.9%) | (98.9%) | (1.1%) | (99.6%) | (0.4%) | (99.3%) | (0.7%) | (100%) | (0%) | (8.4%) | (13.6%) |
| 20 | 1278 | 115 | 891 | 30 | 447 | 5 | 264 | 4 | 161 | 1 | 332 | 302 |
| | (91.7%) | (8.3%) | (96.7%) | (3.3%) | (98.9%) | (1.1%) | (98.5%) | (1.5%) | (99.4%) | (0.6%) | (15.2%) | (23.8%) |
| 25 | 1195 | 198 | 863 | 58 | 436 | 16 | 259 | 9 | 158 | 4 | 529 | 471 |
| | (85.8%) | (14.2%) | (93.7%) | (6.3%) | (96.5%) | (3.5%) | (96.6%) | (3.4%) | (97.5%) | (2.5%) | (24.1%) | (37.1%) |
| 30 | 1095 | 298 | 821 | 100 | 424 | 28 | 252 | 16 | 154 | 8 | 742 | 642 |
| | (78.6%) | (21.4%) | (89.1%) | (10.9%) | (93.8%) | (6.2%) | (94.0%) | (6.0%) | (95.1%) | (4.9%) | (33.9%) | (50.6%) |
| 35 | 1000 | 393 | 769 | 152 | 406 | 46 | 243 | 25 | 153 | 9 | 925 | 773 |
| | (71.8%) | (28.2%) | (83.5%) | (16.5%) | (89.8%) | (10.2%) | (90.7%) | (9.3%) | (94.4%) | (5.6%) | (42.2%) | (60.9%) |
| 40 | 886 | 507 | 701 | 220 | 376 | 76 | 225 | 43 | 145 | 17 | 1101 | 881 |
| | (63.6%) | (36.4%) | (76.1%) | (23.9%) | (83.2%) | (16.8%) | (84.0%) | (16.0%) | (89.5%) | (10.5%) | (50.3%) | (69.4%) |
| 45 | 787 | 606 | 641 | 280 | 341 | 111 | 208 | 60 | 138 | 24 | 1257 | 977 |
| | (56.5%) | (43.5%) | (69.6%) | (30.4%) | (75.4%) | (24.6%) | (77.6%) | (22.4%) | (85.2%) | (14.8%) | (57.4%) | (76.9%) |
| 50 | 672 | 721 | 568 | 353 | 312 | 140 | 188 | 80 | 128 | 34 | 1418 | 1065 |
| | (48.2%) | (51.8%) | (61.7%) | (38.3%) | (69.0%) | (31.0%) | (70.1%) | (29.9%) | (79.0%) | (21.0%) | (64.7%) | (83.9%) |

GG: Grade group
PCa: Prostate cancer
*Patients receiving a biopsy but had a Risk Score below the threshold
**Patients with a negative biopsy or grade group 1 PCa with a Risk Score below the threshold

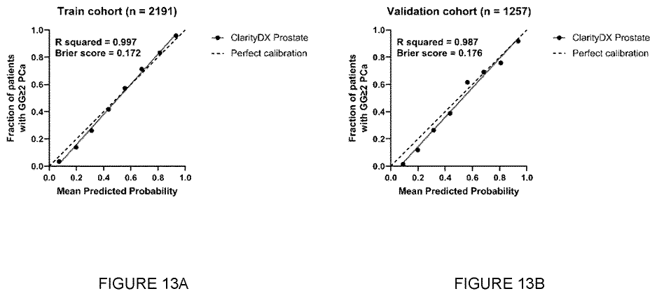

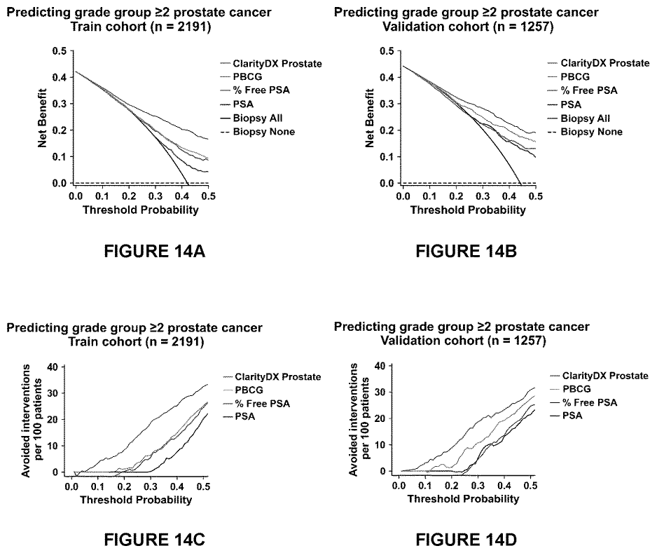

The ClarityDX Prostate model was well calibrated for predicting GG ≥2 prostate cancer with Brier scores of 0.172 and 0.176 for the train and validation cohorts, respectively (FIG. 13A, 13B). FIGS. 13A and 13B shows calibration curves for ClarityDX Prostate model in the train (A) and validation (B) cohorts when predicting grade group ≥2 prostate cancer. Data points fitted with linear regression with R squared values and Brier scores shown. Calibration curves trend close to the perfect calibration line for training and validation cohorts. Linear regression equations fit near the perfectly calibrated line with R squared values of 0.997 and 0.987 for the train and validation cohorts, respectively. Decision curve analysis was performed on ClarityDX Prostate models, PBCG, % free PSA, and PSA. For both the training and validation cohorts, ClarityDX Prostate demonstrated the highest net benefit and reduction in interventions when predicting GG ≥2 prostate cancer using probability thresholds between 0.04 to 0.50, as shown in FIG. 14.

FIG. 14 shows decision curve analysis of ClarityDX Prostate, PBCG, % free PSA, and PSA for predicting grade group ≥2 prostate cancer in the train (A, C) and validation (B, D) cohorts. Net benefit (A, B) and net avoided interventions per 100 patients (C, D) were analyzed assuming interventions required for grade group ≥2 prostate cancer. For almost all probability thresholds, ClarityDX Prostate outperforms the other tests. The recommended ≥25% ClarityDX Prostate threshold is well within the area of highest net benefit for identifying high-risk patients for GG ≥2 prostate cancer.

Using clinical and laboratory data, ClarityDX Prostate can predict the risk of any prostate cancer and clinically significant cancer, defined as GG ≥2, prior to biopsy and in the pre-MRI setting.

Tables 9 and 10 shows performance of the above methods and algorithms (ClarityDX Prostate) with and without DRE, MRI prostate volume and prostate imaging reporting and data system (PI-RADS or PI-RADS score). The performance models were in ensembles of 10 random forest models each calibrated with isotonic regression using cross-validation.

For instance, in one of the training cohorts the clinical indicators used were Age, PSA, free-PSA, and prior negative biopsy i.e. DRE, MRI prostate volume or PI-RADS score were not included, which resulted in an ROC AUC of 0.80. However, in the next set of cohorts, the excluded clinical features were added to evaluate the resulting ROC AUC.

TABLE 9

| | | | | | | | Positive | Negative |
|---|---|---|---|---|---|---|---|---|
| ClarityDX Prostate | Clinical | Patient | ROC | | | | predictive | predictive |
| model features | sites | number | AUC | Threshold | Sensitivity | Specificity | value | value |
| Train cohort | | | | | | | | |
| Age, PSA, free PSA, prior | UCLA, UC, | 2191 | 0.80 | ≥25 | 94 | 34 | 51 | 89 |
| negative biopsy | JHU | | | | | | | |
| Age, PSA, free PSA, prior | UCLA, UC, | 2191 | 0.82 | ≥25 | 94 | 37 | 52 | 89 |
| negative biopsy, DRE | JHU | | | | | | | |
| Age, PSA, free PSA, prior | UCLA, JHU | 1626 | 0.89 | ≥17 | 96 | 45 | 56 | 94 |
| negative biopsy, MRI | | | | | | | | |
| prostate volume, PI-RA [symbol] | | | | | | | | |
| Age, PSA, free PSA, prior | UCLA, JHU | 1626 | 0.89 | ≥17 | 96 | 46 | 56 | 94 |
| negative biopsy, DRE, | | | | | | | | |
| MRI prostate volume,P [symbol] | | | | | | | | |
| Validation cohort | | | | | | | | |
| Age, PSA, free PSA, prior | UA, TUH | 1257 | 0.80 | ≥25 | 95 | 32 | 52 | 89 |
| negative biopsy | | | | | | | | |
| Age, PSA, free PSA, prior | UA, TUH | 1257 | 0.82 | ≥25 | 95 | 35 | 54 | 91 |
| negative biopsy, DRE | | | | | | | | |
| Age, PSA, free PSA, prior | TUH | 317 | 0.87 | ≥17 | 95 | 45 | 50 | 94 |
| negative biopsy, MRI | | | | | | | | |
| prostate volume, PI-RADS | | | | | | | | |
| Age, PSA, free PSA, prior | TUH | 317 | 0.87 | ≥17 | 95 | 47 | 51 | 94 |
| negative biopsy, DRE, | | | | | | | | |
| MRI prostate volume, PI-RAD | | | | | | | | |

UCLA: University of California, Los Angeles
UC: University of Calgary
JHU: Johns Hopkins University
UA: University of Alberta
TUH: Thomayer University Hospital
ROC AUC: Receiver Operating Characteristic Area Under the Curve
DRE: Digital Rectal Exam findings [symbol]
indicates data missing or illegible when filed

TABLE 10

| Features in model | | | | | | | | | |
|---|---|---|---|---|---|---|---|---|---|
| | Prostate | | | | | | | Positive | Negative |
| | volume + | Clinical | Patient | ROC | | | | predictive | predictive |
| DRE | PI-RADS | sites | number | AUC | Threshold | Sensitivity | Specificity | value | value |
| Train cohort | | | | | | | | | |
| No | No | UCLA, UC, | 2191 | 0.80 | ≥25 | 94 | 34 | 51 | 89 |
| | | JHU | | | | | | | |
| Yes | No | UCLA, UC, | 2191 | 0.82 | ≥25 | 94 | 37 | 52 | 89 |
| | | JHU | | | | | | | |
| No | Yes | UCLA, JHU | 1626 | 0.89 | ≥17 | 96 | 45 | 56 | 94 |
| Yes | Yes | UCLA, JHU | 1626 | 0.89 | ≥17 | 96 | 46 | 56 | 94 |
| Validation cohort | | | | | | | | | |
| No | No | UA, TUH | 1257 | 0.80 | ≥25 | 95 | 32 | 52 | 89 |
| Yes | No | UA, TUH | 1257 | 0.82 | ≥25 | 95 | 35 | 54 | 91 |
| No | Yes | TUH | 317 | 0.87 | ≥17 | 95 | 45 | 50 | 94 |
| Yes | Yes | TUH | 317 | 0.87 | ≥17 | 95 | 47 | 51 | 94 |

UCLA: University of California, Los Angeles
UC: University of Calgary
JHU: Johns Hopkins University
UA: University of Alberta
TUH: Thomayer University Hospital
ROC AUC: Receiver Operating Characteristic Area Under the Curve
DRE: Digital Rectal Exam findings

Based on the clinical performance and intended use, the ClarityDX Prostate test is a non invasive, low risk assessment tool that, when used in conjunction with current standard of care practices, may help physicians improve stratification of at-risk patients requiring more expensive diagnostic procedures such as biopsy or MR imaging.

As would be appreciated by a person skilled in the art, one or more illustrative embodiments have been described by way of example only. It will be understood to persons skilled in the art that a number of variations and modifications can be made without departing from the scope of the invention as defined in the claims.

All citations and/or references recited herein are hereby incorporated by reference in their entirety.

### LIST OF REFERENCES

1. Hulsen T. An overview of publicly available patient-centered prostate cancer datasets. Translational andrology and urology. 2019; 8: S64-S77.
2. Fenton J J, Weyrich M S, Durbin S, Liu Y, Bang H, Melnikow J. U.S. Preventive Services Task Force Evidence Syntheses. Prostate-Specific Antigen-Based Screening for Prostate Cancer: A Systematic Evidence Review for the U.S. Preventive Services Task Force. Rockville (M D): Agency for Healthcare Research and Quality (U S); 2018.
3. Alford A V, Brito J M, Yadav K K, Yadav S S, Tewari A K, Renzulli J. The Use of Biomarkers in Prostate Cancer Screening and Treatment. Reviews in Urology. 2017; 19:221-234.
4. Chang E K, Gadzinski A J, Nyame Y A. Blood and urine biomarkers in prostate cancer: Are we ready for reflex testing in men with an elevated prostate-specific antigen? Asian J Urol. 2021; 8:343-353.
5. Heijnsdijk E A, Denham D, de Koning H J. The cost-effectiveness of prostate cancer detection with the use of prostate health index. Value in Health. 2016; 19:153-157.
6. Cooperberg M R, Carroll P R. Trends in management for patients with localized prostate cancer, 1990-2013. Jama. 2015; 314:80-82.
7. Drost F-J H, Rannikko A, Valdagni R, et al. Can active surveillance really reduce the harms of overdiagnosing prostate cancer? A reflection of real life clinical practice in the PRIAS study. Translational andrology and urology. 2018; 7:98-105.
8. Loeb S, Folkvaljon Y, Makarov D V, Bratt O, Bill-Axelson A, Stattin P. Five-year Nationwide Follow-up Study of Active Surveillance for Prostate Cancer. European urology. 2015; 67:233-238.
9. Burdick M J, Reddy C A, Ulchaker J, et al. Comparison of biochemical relapse-free survival between primary Gleason score 3 and primary Gleason score 4 for biopsy Gleason score 7 prostate cancer. International journal of radiation oncology, biology, physics. 2009; 73:1439-1445.
10. Bill-Axelson A, Holmberg L, Garmo H, et al. Radical Prostatectomy or Watchful Waiting in Prostate Cancer-29-Year Follow-up. New England Journal of Medicine. 2018; 379:2319-2329.
11. Prostate Cancer Early Detection. NCCN Guidelines Version 1.2021. Vol 2021: NCCN; 2021.
12. Bosaily A E-S, Parker C, Brown L, et al. PROMIS-prostate M R imaging study: a paired validating cohort study evaluating the role of multi-parametric MRI in men with clinical suspicion of prostate cancer. Contemporary clinical trials. 2015; 42:26-40.
13. Drost F J H, Osses D F, Nieboer D, et al. Prostate MRI, with or without MRI-targeted biopsy, and systematic biopsy for detecting prostate cancer. Cochrane Database of Systematic Reviews. 2019.
14. Parekh D J, Punnen S, Sjoberg D D, et al. A Multi-institutional Prospective Trial in the USA Confirms that the 4Kscore Accurately Identifies Men with High-grade Prostate Cancer. European Urology. 2015; 68:464-470.
15. Ferro M, Bruzzese D, Perdona S, et al. Prostate Health Index (Phi) and Prostate Cancer Antigen 3 (PCA3) significantly improve prostate cancer detection at initial biopsy in a total PSA range of 2-10 ng/ml. PLOS One. 2013; 8: e67687.
16. Van Neste L, Hendriks R J, Dijkstra S, et al. Detection of High-grade Prostate Cancer Using a Urinary Molecular Biomarker-Based Risk Score. Eur Urol. 2016; 70:740-748.
17. Klein E A, Chait A, Hafron J M, et al. The Single-parameter, Structure-based IsoPSA Assay Demonstrates Improved Diagnostic Accuracy for Detection of Any Prostate Cancer and High-grade Prostate Cancer Compared to a Concentration-based Assay of Total Prostate-specific Antigen: A Preliminary Report. Eur Urol. 2017; 72:942-949.
18. McKiernan J, Donovan M J, O'Neill V, et al. A Novel Urine Exosome Gene Expression Assay to Predict High-grade Prostate Cancer at Initial Biopsy. JAMA Oncol. 2016; 2:882-889.
19. Foley R W, Maweni R M, Gorman L, et al. European Randomised Study of Screening for Prostate Cancer (ERSPC) risk calculators significantly outperform the Prostate Cancer Prevention Trial (PCPT) 2.0 in the prediction of prostate cancer: a multi-institutional study. BJU Int. 2016; 118:706-713.
20. Haese A, Trooskens G, Steyaert S, et al. Multicenter Optimization and Validation of a 2-Gene mRNA Urine Test for Detection of Clinically Significant Prostate Cancer Prior to Initial Prostate Biopsy. J Urol. 2019: 101097ju0000000000000293.
21. Van Neste L, Partin A W, Stewart G D, Epstein J I, Harrison D J, Van Criekinge W. Risk score predicts high-grade prostate cancer in DNA-methylation positive, histopathologically negative biopsies. The Prostate. 2016; 76:1078-1087.
22. Kohestani K, Månsson M, Arnsrud Godtman R, et al. The GÖTEBORG prostate cancer screening 2 trial: a prospective, randomised, population-based prostate cancer screening trial with prostate-specific antigen testing followed by magnetic resonance imaging of the prostate. Scand J Urol. 2021; 55:116-124.
23. Sonn G A, Fan R E, Ghanouni P, et al. Prostate Magnetic Resonance Imaging Interpretation Varies Substantially Across Radiologists. European urology focus. 2019; 5:592-599.
24. Westphalen A C, McCulloch C E, Anaokar J M, et al. Variability of the positive predictive value of P I-RADS for prostate MRI across 26 centers: experience of the society of abdominal radiology prostate cancer disease-focused panel. Radiology. 2020; 296:76.
25. Kim S J, Vickers A J, Hu J C. Challenges in adopting level 1 evidence for multiparametric magnetic resonance imaging as a biomarker for prostate cancer screening. JAMA oncology. 2018; 4:1663-1664.
26. Cerantola Y, Dragomir A, Tanguay S, Bladou F, Aprikian A, Kassouf W. Cost-effectiveness of multiparametric magnetic resonance imaging and targeted biopsy in diagnosing prostate cancer. Urologic Oncology: Seminars and Original Investigations. 2016; 34:119.e111-119.e119.
27. Venderink W, Govers™, de Rooij M, Fütterer J J, Sedelaar J M. Cost-effectiveness comparison of imaging-guided prostate biopsy techniques: systematic transrectal ultrasound, direct in-bore MRI, and image fusion. American Journal of Roentgenology. 2017; 208:1058-1063.
28. de Rooij M, Crienen S, Witjes J A, Barentsz J O, Rovers M M, Grutters J P C. Cost-effectiveness of Magnetic Resonance (M R) Imaging and M R-guided Targeted Biopsy Versus Systematic Transrectal Ultrasound-Guided Biopsy in Diagnosing Prostate Cancer: A Modelling Study from a Health Care Perspective. European Urology. 2014; 66:430-436.
29. Loeb S, Vellekoop A, Ahmed H U, et al. Systematic review of complications of prostate biopsy. Eur Urol. 2013; 64:876-892.
30. Pepe P, Aragona F. Morbidity After Transperineal Prostate Biopsy in 3000 Patients Undergoing 12 vs 18 vs More Than 24 Needle Cores. Urology. 2013; 81:1142-1146.
31. Cheng K, Lam W, Chan H, et al. Emergency attendances and hospitalisations for complications after transrectal ultrasound-guided prostate biopsies: a five-year retrospective multicentre study. Hong Kong Medical Journal. 2019; 25:349.
32. Rudzinski J K, Kawakami J. Incidence of infectious complications following transrectal ultrasound-guided prostate biopsy in Calgary, Alberta, Canada: A retrospective population-based analysis. Canadian Urological Association journal=Journal de l′Association des urologues du Canada. 2014; 8: E301-305.
33. Liss M, Ehdaie B, Loeb S, et al. The Prevention and Treatment of the More Common Complications Related to Prostate Biopsy Update. In: Force T, ed. Vol 2022: American Urological Association; 2016.
34. Vasquez C, Kolinsky M, Djebah R, et al. Cohort profile: the Alberta Prostate Cancer Research Initiative (APCaRI) Registry and Biorepository facilitates technology translation to the clinic through the use of linked, longitudinal clinical and patient-reported data and biospecimens from men in Alberta, Canada. BMJ Open. 2020; 10: e037222.
35. World Medical A. World Medical Association Declaration of Helsinki. Ethical principles for medical research involving human subjects. Bulletin of the World Health Organization. 2001; 79:373-374.
36. Athanazio D, Gotto G, Shea-Budgell M, Yilmaz A, Trpkov K. Global Gleason grade groups in prostate cancer: concordance of biopsy and radical prostatectomy grades and predictors of upgrade and downgrade. Histopathology. 2017; 70:1098-1106.
37. DeLong E R, DeLong D M, Clarke-Pearson D L. Comparing the areas under two or more correlated receiver operating characteristic curves: a nonparametric approach. Biometrics. 1988; 44:837-845.
38. Vickers A J, Vertosick E A, Sjoberg D D. Value of a statistical model based on four kallikrein markers in blood, commercially available as 4Kscore, in all reasonable prostate biopsy subgroups. Eur. Urol. 2018; 74.
39. Hupe M C, Philippi C, Roth D, et al. Using PSMA (prostate-specific membrane antigen) evaluation on prostate biopsies for risk stratification at time of initial diagnosis. Journal of Clinical Oncology. 2019; 37:6-6.
40. Ankerst D P, Straubinger J, Selig K, et al. A Contemporary Prostate Biopsy Risk Calculator Based on Multiple Heterogeneous Cohorts. Eur Urol. 2018; 74:197-203.
41. Epstein J I, Zelefsky M J, Sjoberg D D, et al. A Contemporary Prostate Cancer Grading System: A Validated Alternative to the Gleason Score. Eur Urol. 2016; 69:428-435.
42. Ankerst D P, Hoefler J, Bock S, et al. Prostate cancer prevention trial risk calculator 2.0 for the prediction of low-versus high-grade prostate cancer. Urology. 2014; 83:1362-1368.
43. CLSI, Validation of Assays Performed by Flow Cytometry, in CLSI guideline H62. 2021: Wayne, P A.
44. CLSI, Interference Testing in Clinical Chemistry, in CLSI EP07. 2018.
45. Culp, M. B., et al., Recent Global Patterns in Prostate Cancer Incidence and Mortality Rates. Eur Urol, 2020. 77 (1): p. 38-52.
46. Brenner, D. R., et al., Projected estimates of cancer in Canada in 2020. CMAJ, 2020. 192 (9): p. E199-E205.
47. EAU Guidelines. Edn. presented at the EAU Annual Congress 2022, EAU Guidelines Office: Arnhem, The Netherlands.
48. Sung, H., et al., Global Cancer Statistics 2020: GLOBOCAN Estimates of Incidence and Mortality Worldwide for 36 Cancers in 185 Countries. C A: A Cancer Journal for Clinicians, 2021. 71 (3): p. 209-249.
49. Van Poppel, H., et al., Prostate-specific Antigen Testing as Part of a Risk-Adapted Early Detection Strategy for Prostate Cancer: European Association of Urology Position and Recommendations for 2021. European Urology, 2021. 80 (6): p. 703-711.
50. Kimura, T., et al., Global Trends of Latent Prostate Cancer in Autopsy Studies. Cancers (Basel), 2021. 13 (2).
51. Committee, C.C.S.A., Canadian Cancer Statistics 2018. 2018, Canadian Cancer Society: Toronto, O N.
52. Fradet, Y., et al., The burden of prostate cancer in Canada. Canadian Urological Association Journal, 2009. 3 (3 Suppl 2): p. S92-S100.
53. Alford, A. V., et al., The Use of Biomarkers in Prostate Cancer Screening and Treatment. Reviews in Urology, 2017. 19 (4): p. 221-234.
54. Malik, A., S. Srinivasan, and J. Batra, A New Era of Prostate Cancer Precision Medicine. Frontiers in Oncology, 2019. 9.
55. Naji, L., et al., Digital Rectal Examination for Prostate Cancer Screening in Primary Care: A Systematic Review and Meta-Analysis. The Annals of Family Medicine, 2018. 16 (2): p. 149-154.
56. Pepe, P. and F. Aragona, Morbidity After Transperineal Prostate Biopsy in 3000 Patients Undergoing 12 vs 18 vs More Than 24 Needle Cores. Urology, 2013. 81 (6): p. 1142-1146.
57. Efesoy, O., et al., Complications of transrectal ultrasound-guided 12-core prostate biopsy: a single center experience with 2049 patients. Turkish journal of urology, 2013. 39 (1): p. 6-11.
58 Cancer Facts & FIGS. 2022. 2022 [cited 2022 Apr. 17]; Available from: https://www.cancer.org/cancer/prostate-cancer/detection-diagnosis-staging/survival-rates.html #references.
59. Jacklin, C., et al., “More men die with prostate cancer than because of it”—an old adage that still holds true in the 21st century. Cancer Treatment and Research Communications, 2021. 26: p. 100225.
60. Willetts, L., et al., Quantitative Analysis of Human Cancer Cell Extravasation Using Intravital Imaging, in The Tumor Microenvironment: Methods and Protocols, J. Ursini-Siegel and N. Beauchemin, Editors. 2016, Springer New York: New York, N Y. p. 27-37.
61. Klotz, Clinical results of long-term follow-up of a large, active surveillance cohort with localized prostate cancer. 2010.
62. Bell, N., Recommendations on screening for prostate cancer with the prostate-specific antigen test. CMAJ, 2014. 186 (16): p. 1201-2.
63. Ilic, D., et al., Screening for prostate cancer. Cochrane Database of Systematic Reviews, 2013 (1).
64. Draisma, G., et al., Lead Time and Overdiagnosis in Prostate-Specific Antigen Screening: Importance of Methods and Context. JNCI Journal of the National Cancer Institute, 2009. 101 (6): p. 374-383.
65. Welch, H. G. and P. C. Albertsen, Prostate cancer diagnosis and treatment after the introduction of prostate-specific antigen screening: 1986-2005. J Natl Cancer Inst, 2009. 101 (19): p. 1325-9.
66. Tsodikov, A., et al., Reconciling the Effects of Screening on Prostate Cancer Mortality in the ERSPC and PLCO Trials. Ann Intern Med, 2017. 167 (7): p. 449-455.
67. Draisma, G., et al., Lead times and overdetection due to prostate-specific antigen screening: estimates from the European Randomized Study of Screening for Prostate Cancer. J Natl Cancer Inst, 2003. 95 (12): p. 868-78.
68. Brett, A. S. and R. J. Ablin, Prostate-Cancer Screening-What the U.S. Preventive Services Task Force Left Out. New England Journal of Medicine, 2011. 365 (21): p. 1949-1951.
69. Heidenreich, A., et al., EAU guidelines on prostate cancer. Part 1: screening, diagnosis, and treatment of clinically localised disease. Eur Urol, 2011. 59 (1): p. 61-71.
70. Salciccia, S., et al., Biomarkers in Prostate Cancer Diagnosis: From Current Knowledge to the Role of Metabolomics and Exosomes. International journal of molecular sciences, 2021. 22 (9): p. 4367.
Donovan, M. J., et al., A molecular signature of PCA3 and ERG exosomal RNA from non-DRE urine is predictive of initial prostate biopsy result. Prostate Cancer Prostatic Dis, 2015. 18 (4): p. 370-5.
71. Filella, X., et al., Emerging biomarkers in the diagnosis of prostate cancer. Pharmgenomics Pers Med, 2018. 11: p. 83-94.
72. Sanda, M. G., et al., Association Between Combined TMPRSS2: ERG and PCA3 RNA Urinary Testing and Detection of Aggressive Prostate Cancer. JAMA Oncol, 2017. 3 (8): p. 1085-1093.
73. Tosoian, J. J., et al., Use of the MyProstateScore Test to Rule Out Clinically Significant Cancer: Validation of a Straightforward Clinical Testing Approach. Journal of Urology, 2021. 205 (3): p. 732-739.
74. Bronimann, S., et al., An up-to-date catalogue of urinary markers for the management of prostate cancer. Current Opinion in Urology, 2020. 30 (5).
75. Matuszczak, M., J. A. Schalken, and M. Salagierski, Prostate Cancer Liquid Biopsy Biomarkers' Clinical Utility in Diagnosis and Prognosis. Cancers, 2021. 13 (13): p. 3373.
76. Wagaskar, V. G., et al., Clinical Utility of Negative Multiparametric Magnetic Resonance Imaging in the Diagnosis of Prostate Cancer and Clinically Significant Prostate Cancer. European urology open science, 2021. 28: p. 9-16.
77. Maggi, M., et al., SelectMDx and Multiparametric Magnetic Resonance Imaging of the Prostate for Men Undergoing Primary Prostate Biopsy: A Prospective Assessment in a Multi-Institutional Study. Cancers, 2021. 13 (9): p. 2047.
78. Lendínez-Cano, G., et al., Prospective study of diagnostic accuracy in the detection of high-grade prostate cancer in biopsy-naïve patients with clinical suspicion of prostate cancer who underwent the Select MDx test. The Prostate, 2021. 81 (12): p. 857-865.
79. Stovsky, M., et al., Clinical Validation of IsoPSA™, a Single Parameter, Structure Based Assay for Improved Detection of High Grade Prostate Cancer. Journal of Urology, 2019. 201 (6): p. 1115-1120.
80. Zappala, S. M. and Y. Dong, Re: Russo G I, Regis F, Castelli T, et al. A Systematic Review and Meta-analysis of the Diagnostic Accuracy of Prostate Health Index and 4-kallikrein Panel Score in Predicting Overall and High-grade Prostate Cancer. Clin Genitourin Cancer 2017; 15:429-39. Clinical Genitourinary Cancer, 2018. 16 (1): p. e67-e68.
81. Lamy, P.-J., et al., Prognostic Biomarkers Used for Localised Prostate Cancer Management: A Systematic Review. European Urology Focus, 2018. 4 (6): p. 790-803.
82. Habibzadeh, P. and F. Habibzadeh, Re: Eric A. Klein, Arnon Chait, Jason M. Hafron, et al. The Single-parameter, Structure-based IsoPSA Assay Demonstrates Improved Diagnostic Accuracy for Detection of Any Prostate Cancer and High-grade Prostate Cancer Compared to a Concentration-based Assay of Total Prostate-specific Antigen: A Preliminary Report. Eur Urol 2017; 72:942-9. European Urology, 2017. 72 (6): p. e172-e173.
83. Taneja, S. S., Re: Can Urinary PCA3 Supplement PSA in the Early Detection of Prostate Cancer? Journal of Urology, 2015. 194 (1): p. 115-115.
84. Stephan, C., H. Cammann, and K. Jung, Re: Scott A. Tomlins, John R. Day, Robert J. Lonigro, et al. Urine TMPRSS2: ERG Plus PCA3 for Individualized Prostate Cancer Risk Assessment. Eur Urol. In press. http://dx.doi.org/10.1016/j.eururo.2015.04.039. European Urology, 2015. 68 (5): p. e106-e107.
85. Özkan, T. A., Re: Predicting High-Grade Cancer at Ten-Core Prostate Biopsy Using Four Kallikrein Markers Measured in Blood in the ProtecT Study. Journal of Urological Surgery, 2015. 2 (4): p. 200-200.
86. Nordstrom, T., et al., Comparison Between the Four-kallikrein Panel and Prostate Health Index for Predicting Prostate Cancer. European urology, 2015. 68 (1): p. 139-146.
87. Nicholson, A., et al., The clinical effectiveness and cost-effectiveness of the PROGENSA® prostate cancer antigen 3 assay and the Prostate Health Index in the diagnosis of prostate cancer: a systematic review and economic evaluation. Health technology assessment (Winchester, England), 2015. 19 (87): p. i-191.
88. de la Calle, C., et al., Multicenter Evaluation of the Prostate Health Index to Detect Aggressive Prostate Cancer in Biopsy Naïve Men. The Journal of urology, 2015. 194 (1): p. 65-72.
89. Vedder, M. M., et al., The added value of percentage of free to total prostate-specific antigen, PCA3, and a kallikrein panel to the ERSPC risk calculator for prostate cancer in prescreened men. European urology, 2014. 66 (6): p. 1109-1115.
90. Seisen, T., et al., Accuracy of the prostate health index versus the urinary prostate cancer antigen 3 score to predict overall and significant prostate cancer at initial biopsy. The Prostate, 2014. 75 (1): p. 103-111.
91. Partin, A. W., et al., Clinical validation of an epigenetic assay to predict negative histopathological results in repeat prostate biopsies. The Journal of urology, 2014. 192 (4): p. 1081-1087.
92. Catalona, W. J., et al., A multicenter study of [−2]pro-prostate specific antigen combined with prostate specific antigen and free prostate specific antigen for prostate cancer detection in the 2.0 to 10.0 ng/ml prostate specific antigen range. The Journal of urology, 2011. 185 (5): p. 1650-1655.
93. Hessels, D., et al., Predictive value of PCA3 in urinary sediments in determining clinico-pathological characteristics of prostate cancer. The Prostate, 2010. 70 (1): p. 10-16.
94. Hessels, D. and J. A. Schalken, The use of PCA3 in the diagnosis of prostate cancer. Nature Reviews Urology, 2009. 6 (5): p. 255-261.
95. Deras, I. L., et al., PCA3: A Molecular Urine Assay for Predicting Prostate Biopsy Outcome. Journal of Urology, 2008. 179 (4): p. 1587-1592.
96. Andriole, G. L., PCA3 Molecular Urine Assay Correlates With Prostate Cancer Tumor Volume: Implication in Selecting Candidates for Active Surveillance. Yearbook of Urology, 2008. 2008: p. 150.
97. Tomlins, S. A., et al., Recurrent Fusion of TMPRSS2 and ETS Transcription Factor Genes in Prostate Cancer. Science, 2005. 310 (5748): p. 644-648.
98. Haese, A., et al., Multicenter optimization and validation of a 2-gene mRNA urine test for detection of clinically significant prostate cancer before initial prostate biopsy. The Journal of urology, 2019. 202 (2): p. 256-263.
99. Osses, D. F., M. J. Roobol, and I. G. Schoots, Prediction Medicine: Biomarkers, Risk Calculators and Magnetic Resonance Imaging as Risk Stratification Tools in Prostate Cancer Diagnosis. International Journal of Molecular Sciences, 2019. 20 (7): p. 1637.
100. Karlsson, A. A., et al., The cost-effectiveness of prostate cancer screening using the Stockholm3 test. PLOS ONE, 2021. 16 (2): p. e0246674.
101. Shipe, M. E., et al., Developing prediction models for clinical use using logistic regression: an overview. Journal of thoracic disease, 2019. 11 (Suppl 4): p. S574-S584.
102. Alkhateeb, A., G. Atikukke, and L. Rueda, Machine learning methods for prostate cancer diagnosis. Journal of Cancer, 2020. 1 (3): p. 70-75.
103. Passera, R., et al., Machine Learning Techniques in Prostate Cancer Diagnosis According to Prostate-Specific Antigen Levels and Prostate Cancer Gene 3 Score. [symbol] , 2021. 19 (3): p. 164-173.
104. Vedder, M. M., et al., The added value of percentage of free to total prostate-specific antigen, PCA3, and a kallikrein panel to the ERSPC risk calculator for prostate cancer in prescreened men. Eur Urol, 2014. 66 (6): p. 1109-15.
105. Jalali, A., et al., A risk calculator to inform the need for a prostate biopsy: a rapid access clinic cohort. BMC Medical Informatics and Decision Making, 2020. 20 (1): p. 148.
106. Louie, K. S., et al., Do prostate cancer risk models improve the predictive accuracy of PSA screening? A meta-analysis. Ann Oncol, 2015. 26 (5): p. 848-64.

What is claimed is:

1. A method for predicting a possibility of developing clinically significant prostate cancer comprising:
a) statistically converting one or more clinical data points of a plurality of patients;
b) feeding the one or more statistically converted clinical data points into a prediction system;
c) training the prediction system using a software module, wherein the software module trains the prediction system to create a probability model using the one or more statistically converted clinical data points; and
d) predicting the possibility of developing clinically significant prostate cancer using the probability model created by the prediction system.

2. The method of claim 1, wherein statistically converting one or more clinical data points further comprises:
a) log-transforming one or more clinical data points;
b) obtaining a ratio of one or more clinical data points; or
c) standardizing the value of one or more clinical data points or one or more statistically converted clinical data points.

3. The method of claim 1, wherein creating the probability model further comprises calculating a Risk Score using the one or more statistically converted clinical data points to predict the possibility of developing clinically significant prostate cancer.

4. The method of claim 3, wherein calculating the Risk Score further comprises:
a) recommending immediate clinical evaluation, biopsy and/or medical intervention if the Risk Score is high; and
b) recommending future clinical evaluation and/or medical intervention if the Risk Score is low.

5. The method of claim 1, further comprising the step of testing the probability model created by the prediction system for accuracy using one or more pre-defined metric, wherein the pre-defined metric is diagnostic specificity, diagnostic sensitivity, diagnostic positive predictive value, diagnostic negative predictive value, or a combination thereof.

6. The method of claim 1, further comprising the step of grid searching through the one or more clinical data points to determine an ideal combination of clinical data points, wherein the ideal combination of clinical data points are used to improve the probability model created by the prediction system.

7. The method of claim 1, wherein one or more clinical data points are selected from the group consisting of age, biopsy result, digital rectal exam (DRE) result, total prostate specific antigen (PSA) level, free PSA level, number of prior negative biopsies, prostate volume, ethnicity, medication use around the time of biopsy, result of a blood-based prostate cancer diagnostic assay, result of a tissue-based prostate cancer diagnostic assay, result of a urine-based prostate cancer diagnostic assay, prostate imaging reporting and data system score (PI-RADS) or a combination thereof.

8. The method of claim 7, wherein:
a) the total PSA level is statistically converted or log-transformed before feeding it into the prediction system; or
b) wherein the free PSA level is statistically converted or transformed to free PSA ratio before feeding it into the prediction system; or
c) wherein the age is statistically converted before feeding it into the prediction system, wherein optionally the age is converted to obtain an age-related risk of grade group ≥2 prostate cancer before feeding it into the prediction system.

9. The method of claim 8, wherein the free PSA level is transformed to free PSA ratio by dividing the free PSA level with the total PSA level, wherein optionally the free PSA ratio is converted to a free PSA ratio below a linear value, wherein the linear value is the value of free PSA ratio at which the Risk Score starts showing variability, wherein optionally the linear value is in the range of 0.1-0.5.

10. The method of claim 9, wherein the free PSA ratio is converted to the free PSA ratio below a linear value using an equation:
$$free PSA ratio below a linear value = max ( linear value - free PSA ratio , 0 )$$

11. The method of claim 9, wherein the free PSA ratio is converted to a free PSA ratio below 0.3 using an equation:
$$free PSA ratio below 0.3 = max ( 0 \text{.3} - free PSA ratio , 0 )$$
wherein 0.3 is the value of free PSA ratio at which the Risk Score starts showing variability.

12. The method of claim 2, wherein the one or more clinical data points or the one or more statistically converted clinical data points are standardized to obtain a standardized feature value (Z-score) for each clinical data point using an equation:
$$Standardized feature value = \frac{clinical data point value - clinical data point mean value}{clinical data point standard deviation value}$$

13. The method of claim 1, further comprising the step of determining diagnostic ability of the prediction system by plotting an Area Under the Curve (AUC) Receiver Operating Characteristic (ROC) curve.

14. The method of claim 1, wherein the probability model is a logistic regression model and/or a neural network model.

15. The method of claim 1, wherein the prediction system is a computer, an operating system, a software, or a machine learning algorithm.

16. The method of claim 1, wherein the software module trains the logistic regression system by optimizing a set of model hyperparameters to obtain a favorable AUC ROC value using the one or more clinical data points, wherein the model hyperparameters are optimized by grid searching through the model hyperparameters individually and/or in combination.

17. A non-transitory computer readable medium having stored thereon an algorithm that, when executed by a processor, cause the processor to perform the method for predicting a possibility of developing clinically significant prostate cancer, wherein the algorithm performs the method steps of any one of claims 1-16.

18. A kit for predicting a possibility of developing clinically significant prostate cancer comprising:
a) the non-transitory computer readable medium as defined in claim 17; and
b) instructions for performing the method for predicting the possibility of developing clinically significant prostate cancer according to any one of claims 1-16.

19. A software as a medical device for predicting the possibility of developing clinically significant prostate cancer, wherein the software is trained to perform the method according to any one of claims 1-16.

20. A system comprising the non-transitory computer readable medium of claim 17 for predicting a possibility of developing clinically significant prostate cancer.

21. A method for predicting risk of developing clinically significant prostate cancer comprising:
a) processing one or more clinical data points obtained from a plurality of patients, wherein the plurality of patients are selected based on a set of pre-determined criteria;
b) optimizing one or more model hyper parameters by a grid searching technique;
c) feeding the processed clinical data points and the optimized model hyper parameters into a prediction system;
d) training the prediction system to generate a risk score using the processed clinical data points and the model hyper parameters using a machine learning module; and
e) predicting the risk of developing clinically significant prostate cancer using the risk score generated by the prediction system.

22. The method of claim 21, wherein the set of pre-determined criteria for selecting the plurality of patients comprise:
a) patient age;
b) prior prostate cancer diagnosis;
c) referral for biopsy;
d) total prostate specific antigen (total PSA) result;
e) prostate volume;
f) digital rectal exam result;
g) PI-RADS score or
h) a combination thereof.

23. The method of claim 21 or 22, wherein the processing step comprises:
a) scaling the one or more clinical data points by calculating a z-score;
b) performing median feature value imputation on the one or more clinical data points;
c) statistically converting the one or more clinical data points;
d) determining threshold value for the one or more clinical data points by calculating percentage sensitivity for clinically significant prostate cancer;
e) determining confidence intervals for the one or more clinical data points by bias-correction and accelerated bootstrapping;
f) performing decision curve analysis on the one or more clinical data points; or
g) determining statistical significance of the one more clinical data points by calculating p-values ≤0.05.

24. The method of any one of claims 21-23, wherein the one or more clinical data points are total PSA (ng/ml), free PSA (ng/ml), % free PSA (100*free PSA/total PSA), age (years), Caucasian (yes or no), African American (yes or no), Asian (yes or no), Native American (yes or no), Latin (yes or no), digital rectal exam (DRE) findings (normal or abnormal), family history of prostate cancer, previous negative biopsy (yes or no), or the number of previous negative biopsies.

25. The method of claim 23, wherein the step of statistically converting one or more clinical data points comprise log-transforming the one or more clinical data points, wherein optionally the clinical data point is total PSA.

26. The method of claim 25, wherein statistically converting one or more clinical data points further comprises standardizing the value of one or more clinical data points or one or more statistically converted clinical data points.

27. The method of claim 3, wherein the statistically converting one or more clinical data points comprise engineering the one or more clinical data points by quantile grouping, wherein optionally the clinical data point is age.

28. The method of claim 27, wherein the quantile grouping comprises plotting the clinical data point in each quantile against the probability of developing clinically significant prostate cancer.

29. The method of any one of claims 21-28, wherein the optimizing step comprises grid searching through various combinations of the model hyper parameters to determine a receiver operating characteristic area under the curve (ROC AUC) value, optionally by using a five-fold cross-validation.

30. The method of any one of claims 21-29, wherein the training step comprises training the prediction system to predict the risk of developing clinically significant prostate cancer from a previous biopsy result.

31. The method of any one of claims 21-30, wherein the prediction system is logistic regression, linear discriminant analysis, quadratic discriminant analysis, k-nearest neighbors, linear basis function support vector machines, radial basis function support vector machines, single decision tree, random forest, lightGBM, XGBoost, or multilayer perceptron.

32. The method of any one of claims 21-31, wherein the method further comprises a step of improving the performance of the prediction system by using different subsets of training data and calibrating it with isotonic regression.

33. The method of claim 32, wherein the accuracy of the prediction system is ascertained by determining an accuracy metric such as diagnostic specificity, diagnostic sensitivity, diagnostic positive predictive value, diagnostic negative predictive value, diagnostic ability or a combination thereof.

34. The method of any one of claims 21-33, wherein the diagnostic specificity of the prediction system is determined by using a risk score threshold of ≥25%.

35. The method of claim 34, wherein the method further comprises:
a) recommending immediate clinical evaluation, biopsy and/or medical intervention if the risk score is high based on the risk score threshold of ≥25%; and
b) recommending future clinical evaluation if the risk score is low based on the risk score threshold of ≥25%.

36. The method of any one of claims 21-35, wherein the diagnostic ability of the prediction system is determined by plotting a plurality of ROC AUC curves.

37. The method of any one of claims 31-36, the method further comprises comparing the accuracy metric of the prediction system with an accuracy metric of a third party risk calculator or a previous biopsy result.

38. The method of any one of claims 31-37, wherein the prediction system is a computer, an operating system, a software, or a machine learning algorithm.

39. A non-transitory computer readable medium having stored thereon an algorithm that, when executed by a processor, cause the processor to perform the method for predicting the risk of developing clinically significant prostate cancer, wherein the algorithm performs the method of any one of claims 21-38.

40. A kit for predicting risk of developing clinically significant prostate cancer comprising:
a) the non-transitory computer readable medium of claim 39; and
b) instructions for performing the method for predicting the risk of developing clinically significant prostate cancer according to any one of claims 21-38.

41. A software as a medical device for predicting the risk of developing clinically significant prostate cancer, wherein the software is trained to perform the method according to any one of claims 21-38.

42. A system comprising the non-transitory computer readable medium of claim 39 for predicting the risk of developing clinically significant prostate cancer.

43. Use of the system of claim 42, as an early diagnostic tool a) to predict or determine the risk of developing clinically significant prostate cancer; or b) to identify patients with a higher risk of developing clinically significant prostate cancer.
